SEQUENCE LISTING (18 sequences)

SEQ ID NO: 1 - DNA, 1509 bp, Klebsiella pneumoniae
  source 1..1509: mol_type = other DNA
  CDS 1..1509: protein_id = 2; translation = MTAPIQDLRDAIALLQQHDNQYLETDHPVDPNAELAGVYRHIGA GGTVKRPTRIGPAMMFNNIKGYPHSRILVGMHASRQRAALLLGCEASQLALEVGKAVK KPVAPVVVPASSAPCQEQIFLADDPDFDLRTLLPAPTNTPIDAGPFFCLGLALASDPV DASLTDVTIHRLCVQGRDELSMFLAAGRHIEVFRQKAEAAGKPLPITINMGLDPAIYI GACFEAPTTPFGYNELGVAGALRQRPVELVQGVSVPEKAIARAEIVIEGELLPGVRVR EDQHTNSGHAMPEFPGYCGGANPSLPVIKVKAVTMRNNAILQTLVGPGEEHTTLAGLP TEASIWNAVEAAIPGFLQNVYAHTAGGGKFLGILQVKKRQPADEGRQGQAALLALATY SELKNIILVDEDVDIFDSDDILWAMTTRMQGDVSITTIPGIRGHQLDPSQTPEYSPSI RGNGISCKTIFDCTVPWALKSHFERAPFADVDPRPFAPEYFARLEKNQGSAK
atgaccgcaccgattcaggatctgcgcgacgccatcgcgctgctgcaacagcatgacaatcagtatctcgaaaccgatcatccggttgaccctaacgccgagctggccggtgtttatcgccatatcggcgcgggcggcaccgtgaagcgccccacccgcatcgggccggcgatgatgtttaacaatattaagggttatccacactcgcgcattctggtgggtatgcacgccagccgccagcgggccgcgctgctgctgggctgcgaagcctcgcagctggcccttgaagtgggtaaggcggtgaaaaaaccggtcgcgccggtggtcgtcccggccagcagcgccccctgccaggaacagatctttctggccgacgatccggattttgatttgcgcaccctgcttccggcgcccaccaacacccctatcgacgccggccccttcttctgcctgggcctggcgctggccagcgatcccgtcgacgcctcgctgaccgacgtcaccatccaccgcttgtgcgtccagggccgggatgagctgtcgatgtttcttgccgccggccgccatatcgaagtgtttcgccaaaaggccgaggccgccggcaaaccgctgccgataaccatcaatatgggtctcgatccggccatctatattggcgcctgcttcgaagcccctaccacgccgttcggctataatgagctgggcgtcgccggcgcgctgcgtcaacgtccggtggagctggttcagggcgtcagcgtcccggagaaagccatcgcccgcgccgagatcgttatcgaaggtgagctgttgcctggcgtgcgcgtcagagaggatcagcacaccaatagcggccacgcgatgccggaatttcctggctactgcggcggcgctaatccgtcgctgccggtaatcaaagtcaaagcagtgaccatgcgaaacaatgcgattctgcagaccctggtgggaccgggggaagagcataccaccctcgccggcctgccaacggaagccagtatctggaatgccgtcgaggccgccattccgggctttttacaaaatgtctacgcccacaccgcgggtggcggtaagttcctcgggatcctgcaggtgaaaaaacgtcaacccgccgatgaaggccggcaggggcaggccgcgctgctggcgctggcgacctattccgagctaaaaaatattattctggttgatgaagatgtcgacatctttgacagcgacgatatcctgtgggcgatgaccacccgcatgcagggggacgtcagcattacgacaatccccggcattcgcggtcaccagctggatccgtcccagacgccggaatacagcccgtcgatccgtggaaatggcatcagctgcaagaccatttttgactgcacggtcccctgggcgctgaaatcgcactttgagcgcgcgccgtttgccgacgtcgatccgcgtccgtttgcaccggagtatttcgcccggctggaaaaaaaccagggtagcgcaaaataa

SEQ ID NO: 2 - protein, 502 aa, Klebsiella pneumoniae
MTAPIQDLRDAIALLQQHDNQYLETDHPVDPNAELAGVYRHIGAGGTVKRPTRIGPAMMFNNIKGYPHSRILVGMHASRQRAALLLGCEASQLALEVGKAVKKPVAPVVVPASSAPCQEQIFLADDPDFDLRTLLPAPTNTPIDAGPFFCLGLALASDPVDASLTDVTIHRLCVQGRDELSMFLAAGRHIEVFRQKAEAAGKPLPITINMGLDPAIYIGACFEAPTTPFGYNELGVAGALRQRPVELVQGVSVPEKAIARAEIVIEGELLPGVRVREDQHTNSGHAMPEFPGYCGGANPSLPVIKVKAVTMRNNAILQTLVGPGEEHTTLAGLPTEASIWNAVEAAIPGFLQNVYAHTAGGGKFLGILQVKKRQPADEGRQGQAALLALATYSELKNIILVDEDVDIFDSDDILWAMTTRMQGDVSITTIPGIRGHQLDPSQTPEYSPSIRGNGISCKTIFDCTVPWALKSHFERAPFADVDPRPFAPEYFARLEKNQGSAK

SEQ ID NO: 3 - DNA, 1509 bp, synthetic construct
  source 1..1509: mol_type = other DNA
  CDS 1..1509: protein_id = 4; translation = MTAPIQDLRDAIALLQQHDNQYLETDHPVDPNAELAGVYRHIGA GGTVKRPTRIGPAMMFNNIKGYPHSRILVGMHASRQRAALLLGCEASQLALEVGKAVK KPVAPVVVPASSAPCQEQIFLADDPDFDLRTLLPAPTNTPIDAGPFFCLGLALASDPV DASLTDVTIHRLCVQGRDELSMFLAAGRHIEVFRQKAEAAGKPLPITINMGLDPAIYI GACFEAPTTPFGYNELGVAGALRQRPVELVQGVSVPEKAIARAEIVIEGELLPGVRVR EDQHTNSGHAMPEFPGYCGGANPSLPVIKVKAVTMRNNAILQTLVGPGEEHTTLAGLP TEASIWNAVEAAIPGFLQNVYAHTAGGGKFLGILQVKKRQPADEGRQGQAALLALATY SELKNIILVDEDVDIFDSDDILWAMTTRMQGDVSITTIPGIRGHQLDPSQTPEYSPSI RGNGISCKTIFDCTVPWALKSHFERAPFADVDPRPFAPEYFARLEKNQGSAK
atgaccgcaccgattcaggatctgcgtgatgcaattgccctgctgcaacagcatgataatcagtatctggaaaccgatcatccggttgatccgaatgcagaactggcaggcgtttatcgtcatattggtgccggtggcaccgttaaacgtccgacacgtattggtccggcaatgatgtttaataacattaaaggttatccgcacagccgtattctggttggtatgcatgcaagccgtcagcgtgcagcactgctgctgggttgtgaagcaagtcagctggcactggaagttggtaaagcagttaaaaaaccggttgcaccggtggttgttccggcaagcagcgcaccgtgtcaagagcagatttttctggcagatgatccggattttgatctgcgtaccctgctgcctgcaccgaccaatacaccgattgatgcaggtccgtttttttgtctgggtttagcactggcaagcgatccggtggatgcaagcctgaccgatgttaccattcatcgtctgtgtgttcagggtcgtgatgaactgagcatgttcctggcagcaggtcgccatattgaagtttttcgtcagaaagcagaagcagcaggtaaaccgctgccgattaccattaatatgggtttagatccggcaatctatatcggtgcatgttttgaagcaccgacaacaccgtttggttataatgaactgggtgttgcgggtgcactgcgtcagcgtccggttgaactggttcagggtgttagcgttccggaaaaagcaattgcacgtgccgaaattgttattgaaggtgaactgttaccgggtgttcgtgttcgtgaagatcagcataccaatagcggtcatgcaatgccggaatttccgggttattgtggtggtgcaaatccgagcctgccggttattaaagttaaagccgttaccatgcgcaataatgcaattctgcagaccctggttggtccgggtgaagaacataccacactggcaggtctgccgaccgaagcaagcatttggaatgcagttgaagcagcaattccgggtttcctgcagaatgtttatgcacataccgcaggcggtggtaaatttctgggtatcctgcaggtaaaaaaacgtcagcctgcagatgaaggtcgtcagggtcaagcagccctgctggccctggcaacctatagcgaactgaaaaatatcattctggtggatgaggatgtggacatttttgatagtgatgatattctgtgggcaatgaccacacgtatgcagggtgatgttagcattaccaccattccgggtattcgcggtcatcagctggatccgagccagacaccggaatattcaccgagcattcgtggtaatggtattagctgcaaaaccatctttgattgtaccgttccgtgggcactgaaaagccattttgaacgtgcaccgtttgcagatgttgatccgcgtccgtttgcacctgaatattttgcacgtctggaaaaaaatcagggtagcgcaaaataa

SEQ ID NO: 4 - protein, 502 aa, synthetic construct; identical to SEQ ID NO: 2

SEQ ID NO: 5 - DNA, 570 bp, Escherichia coli
  source 1..570: mol_type = other DNA
  CDS 1..570: protein_id = 6; translation = MKRLIVGISGASGAIYGVRLLQVLRDVTDIETHLVMSQAARQTL SLETDFSLREVQALADVTHDARDIAASISSGSFQTLGMVILPCSIKTLSGIVHSYTDG LLTRAADVVLKERRPLVLCVRETPLHLGHLRLMTQAAEIGAVIMPPVPAFYHRPQSLD DVINQTVNRVLDQFAITLPEDLFARWQGA
atgaaacgactcattgtaggcatcagcggtgccagcggcgcgatttatggcgtgcgcttattacaggttctgcgcgatgtcacagatatcgaaacgcatctggtgatgagccaggcagcgcgccagaccttatccctcgaaacggatttttctctgcgcgaagtgcaggcattagccgatgtcacgcacgatgcgcgcgatattgccgccagcatctcttccggttctttccagacgctggggatggtgattttaccctgttcaatcaaaaccctttccggcattgtccatagctatactgatggcttactgacccgtgcggcagatgtggtgctgaaagagcgtcgcccgttggtgctctgcgtgcgtgaaacaccattgcacttaggccatctgcgtttaatgactcaggcggcagaaatcggtgcggtgattatgcctcccgttccggcgttttatcatcgcccgcaatcccttgatgatgtgataaatcagacggttaatcgtgttcttgaccagtttgcgataacccttcctgaagatctctttgcccgctggcagggcgcataa

SEQ ID NO: 6 - protein, 189 aa, Escherichia coli
MKRLIVGISGASGAIYGVRLLQVLRDVTDIETHLVMSQAARQTLSLETDFSLREVQALADVTHDARDIAASISSGSFQTLGMVILPCSIKTLSGIVHSYTDGLLTRAADVVLKERRPLVLCVRETPLHLGHLRLMTQAAEIGAVIMPPVPAFYHRPQSLDDVINQTVNRVLDQFAITLPEDLFARWQGA

SEQ ID NO: 7 - DNA, 1488 bp, Bacillus sp.
  source 1..1488: mol_type = other DNA
  CDS 1..1488: protein_id = 8; translation = MSEKMNKVTDLRSALELLKSIPGQLIETDEPVNPQAELSGVYRH VGAGGTVMRPTKIGPAMVFNHVLGHEDSSVVIGLLASRERVGLMLDTKPERLGFLLNE AVKNPIDPITISNDKAKAQEVVHYAEDPDFDIRKLIPAPTNTEEDAGPYITIGMCYAS DSETGESDITIHRLCLQSKDEMTMYFVPGRHLEVFREKAEKAGKPLPISISIGVDPAI EVAACFEAPTTPLGFNELSVAGAIRKEAVELVQCLTIDEKAIANAEYVIEGELVPGVR VREDQNTNTGKAMPEFPGYTGSAVADLPLIKVKAVTHRVNPIMQTVIGPSEEHVNMAG IPTEASILQMVEKSMPGKLLNVYAHTSGGGKYMAVLQFKKSQPSDEGRQRQAALLAFS AFSELKHVFIVDEDVDPFDSNDVLWALNTRYQGDVDTVFIPGVRCHPLDPTQDPAYSP TIQEKGISCKTIFDCTVPFHLKDEFKRSEFKEVDPSRFVPGFSNS
atgtctgaaaaaatgaacaaagtgacagatttacgttctgcgttggaactcctgaaatccattccaggacaattgattgaaacggatgaaccagtcaatcctcaagctgaattatctggggtttatcgtcatgtcggtgctggcggaacagtcatgcgtccaacaaaaatcggacctgcaatggttttcaatcatgtattgggacatgaagattcaagcgtggtgatcggacttttagccagcagggaaagagtcggcctcatgctggatacaaaaccggagcgactgggattccttttaaatgaagctgtaaaaaatccaattgatccaatcaccatttcaaatgataaagcgaaggctcaggaagtggttcactatgcagaagatccggatttcgatatcagaaaattaatacctgcgcctacgaatacagaggaagatgcaggcccgtatatcacgataggcatgtgctatgcctccgattcagaaacgggtgaatctgatattacgatacatcgtttatgtttacaaagtaaagatgagatgaccatgtattttgttcctggacgtcatttggaagtttttcgtgaaaaggctgaaaaagcaggaaaaccgttacccatttccattagtatcggtgttgaccctgctattgaagtcgctgcttgttttgaagcaccaacgacaccccttggctttaatgagttgagtgttgcaggggcgatcaggaaagaagcggtagaattggtccaatgcttgacgattgatgaaaaggctatcgcgaatgctgaatatgtaatagaaggggaattggttcccggagtgcgtgttcgggaagatcaaaacaccaatacgggaaaagcaatgccggaattccccggttatacaggttcagctgttgcggatctgccactaatcaaggtgaaagccgtaacgcaccgagtcaacccaatcatgcaaacagttattggtccgagtgaggagcatgtgaatatggctggcattccaactgaagcaagtattctgcaaatggtggaaaagtccatgccaggaaaattattgaatgtatatgcccatacttcaggaggaggtaaatacatggctgttcttcaattcaagaaaagtcagccaagtgatgaagggcgtcaaagacaagctgcactattggcgttctccgccttttctgaactgaagcatgtatttatcgtggatgaagatgtggatcccttcgatagcaatgatgttttatgggcgttaaatacccgttaccaaggtgatgttgatacggttttcattcctggagtgcgctgtcacccgttagatcctacacaagatcccgcatacagccctaccattcaggaaaaggggatttcctgtaaaaccatatttgactgcacagtaccttttcatttaaaagatgaatttaaaaggtctgaatttaaagaggtggatccaagtcgattcgttcctggattttccaattcataa

SEQ ID NO: 8 - protein, 495 aa, Bacillus sp.
MSEKMNKVTDLRSALELLKSIPGQLIETDEPVNPQAELSGVYRHVGAGGTVMRPTKIGPAMVFNHVLGHEDSSVVIGLLASRERVGLMLDTKPERLGFLLNEAVKNPIDPITISNDKAKAQEVVHYAEDPDFDIRKLIPAPTNTEEDAGPYITIGMCYASDSETGESDITIHRLCLQSKDEMTMYFVPGRHLEVFREKAEKAGKPLPISISIGVDPAIEVAACFEAPTTPLGFNELSVAGAIRKEAVELVQCLTIDEKAIANAEYVIEGELVPGVRVREDQNTNTGKAMPEFPGYTGSAVADLPLIKVKAVTHRVNPIMQTVIGPSEEHVNMAGIPTEASILQMVEKSMPGKLLNVYAHTSGGGKYMAVLQFKKSQPSDEGRQRQAALLAFSAFSELKHVFIVDEDVDPFDSNDVLWALNTRYQGDVDTVFIPGVRCHPLDPTQDPAYSPTIQEKGISCKTIFDCTVPFHLKDEFKRSEFKEVDPSRFVPGFSNS

SEQ ID NO: 9 - DNA, 1488 bp, synthetic construct
  source 1..1488: mol_type = other DNA
  CDS 1..1488: protein_id = 10; translation = MSEKMNKVTDLRSALELLKSIPGQLIETDEPVNPQAELSGVYRH VGAGGTVMRPTKIGPAMVFNHVLGHEDSSVVIGLLASRERVGLMLDTKPERLGFLLNE AVKNPIDPITISNDKAKAQEVVHYAEDPDFDIRKLIPAPTNTEEDAGPYITIGMCYAS DSETGESDITIHRLCLQSKDEMTMYFVPGRHLEVFREKAEKAGKPLPISISIGVDPAI EVAACFEAPTTPLGFNELSVAGAIRKEAVELVQCLTIDEKAIANAEYVIEGELVPGVR VREDQNTNTGKAMPEFPGYTGSAVADLPLIKVKAVTHRVNPIMQTVIGPSEEHVNMAG IPTEASILQMVEKSMPGKLLNVYAHTSGGGKYMAVLQFKKSQPSDEGRQRQAALLAFS AFSELKHVFIVDEDVDPFDSNDVLWALNTRYQGDVDTVFIPGVRCHPLDPTQDPAYSP TIQEKGISCKTIFDCTVPFHLKDEFKRSEFKEVDPSRFVPGFSNS
atgagcgagaaaatgaacaaagtgaccgatctgcgtagcgcactggaactgctgaaaagcattccgggtcagctgattgaaaccgatgaaccggttaatccgcaggcagaactgagcggtgtttatcgtcatgttggtgccggtggcaccgttatgcgtccgaccaaaattggtccggcaatggtttttaatcatgtgctgggtcatgaagatagcagcgttgttattggtctgctggcaagccgtgaacgtgttggtctgatgctggataccaaaccggaacgtctgggttttctgctgaatgaagcagttaaaaatccgattgatccgatcaccatcagcaacgataaagcaaaagcacaagaggttgttcactatgcagaagatccggattttgatatccgtaaactgattccggcaccgaccaataccgaagaagatgcaggtccgtatattaccattggtatgtgttatgcaagcgatagcgaaaccggtgaaagcgatattacaattcatcgtctgtgtctgcagagcaaagatgaaatgaccatgtattttgttccgggtcgtcatctggaagtttttcgtgaaaaagcagaaaaagccggtaaaccgctgccgattagcattagcattggtgttgatccggcaattgaagttgcagcatgttttgaagcaccgacaacaccgctgggttttaatgaactgagtgttgccggtgcaattcgtaaagaagcagttgaactggttcagtgtctgaccattgatgaaaaagcgattgcaaatgccgaatatgtgattgaaggtgaactggtgcctggtgttcgtgttcgtgaagatcagaataccaatacaggtaaagcaatgccggaatttccgggttataccggtagcgcagttgccgatctgccgctgattaaagttaaagcagttacccatcgtgtgaacccgattatgcagaccgtgattggtccgagcgaagaacatgttaatatggcaggtattccgaccgaagcaagcattctgcagatggttgaaaaatcaatgcctggtaaactgcttaacgtttatgcacataccagcggtggtggcaaatatatggcagttctgcagtttaagaaaagccagccgagtgatgaaggtcgtcagcgtcaggcagcactgctggcatttagcgcctttagcgaactgaaacatgtgtttattgtggatgaagatgtggatccgtttgatagcaatgatgttctgtgggcactgaatacccgttatcagggtgatgttgataccgtttttattccgggtgttcgttgtcatccgctggatccgacacaggatccggcatatagcccgaccattcaagaaaaaggtattagctgcaaaaccatctttgattgcaccgttccgtttcacctgaaagatgaatttaaacgcagcgagtttaaagaggttgatccgagccgttttgttcctggttttagcaatagctaa

SEQ ID NO: 10 - protein, 495 aa, synthetic construct; identical to SEQ ID NO: 8

SEQ ID NO: 11 - DNA, 1473 bp, Lactiplantibacillus pentosus
  source 1..1473: mol_type = other DNA
  CDS 1..1473: protein_id = 12; translation = MAEQPWDLRRVLDEIKDDPKNYHETDVEVDPNAELSGVYRYIGA GGTVERPTQEGPAMMFNNVKGFPDTRVLTGLMASRRRVGKMFHHDYQTLGQYLNDAVS NPVAPETVAEKDAPAHEVIYKSTDEGFDIRKLVAAPTNTPQDAGPYITVGVVFGSSMD KSKSDVTIHRMVLEDKDKLGIYIMPGGRHIGAFAEEYEKANKPMPITINIGLDPAITI GATFEPPTTPFGYNELGVAGAIRNQAVQLVDGVTVDEKAIARSEYTLEGYIMPNERIQ EDINTHTGKAMPEFPGYDGDANPALQVIKVTAVTHRENAIMQSVIGPSEEHVSMAGIP TEASILQLVNRAIPGKVTNVYNPPAGGGKLMTIMQIHKDNEADEGIQRQAALLAFSAF KELKTVILVDEDVDIFDMNDVIWTMNTRFQADQDLMVLSGMRNHPLDPSERPQYDPKS IRFRGMSSKLVIDGTVPYDMKDQFERAQFMKVDDWEKYLK
atggcagaacaaccatgggatttacgccgggtacttgatgagatcaaagatgatcccaagaactatcatgaaaccgacgttgaagtagatccaaatgcggaactttctggtgtttatcggtatattggtgctggtgggaccgttgaacggccaacacaagaaggtccagcaatgatgttcaacaacgtgaagggttttcctgacacgcgtgtcttgactggtttaatggctagccgtcgtcgggttggtaagatgttccatcatgattaccaaacgttaggtcaatatcttaacgatgcagtttcaaatccagttgcaccggaaacggtcgctgaaaaggatgcaccggctcacgaagtcatttacaagtcaactgatgaaggctttgatattcggaagttagttgcagcgccaactaatacgccacaagatgctggtccatatatcacggtcggtgttgtcttcggttcaagcatggacaagtccaagagtgacgttacgattcaccggatggttcttgaagacaaggacaagctcggaatctacatcatgcctggtggccgtcatatcggtgcctttgccgaagaatatgaaaaggccaataaaccaatgccaatcacgatcaacattggtttggatcctgccattaccattggtgccacctttgaaccacctaccaccccatttggttacaacgaattaggggttgctggtgccattcggaatcaagccgttcaattagtcgatggggttaccgttgatgaaaaggcgattgcacgttctgaatacacgttggaaggctacatcatgcctaacgaacggattcaagaagacatcaacacccatacgggtaaagcgatgcctgaattcccaggttatgatggtgatgctaacccagccttacaagtcatcaaggtgacggcggtaacgcatcgggaaaatgccatcatgcaaagtgttatcggaccatccgaagaacatgtcagcatggctggtattccaaccgaagcaagtatcttacagttagttaaccgcgccattcctggaaaagtgacgaacgtttacaatccgccggctggtggtggtaagttgatgaccatcatgcagattcataaggataatgaagcggatgaagggattcaacggcaagcagccttattggcgttctcagcgttcaaggaattgaagacggtcattttagttgatgaagacgttgatattttcgatatgaacgatgtgatttggaccatgaatacccgtttccaagctgaccaagacttgatggtcttatccggtatgcggaaccatccattggatccatcagaacggccacaatacgatccaaaatcgattcgtttccgtgggatgagttcgaaattggttatcgatggcactgtgccatacgatatgaaagaccaatttgaacgggctcaattcatgaaggtcgatgactgggaaaagtatttgaaataa

SEQ ID NO: 12 - protein, 490 aa, Lactiplantibacillus pentosus
MAEQPWDLRRVLDEIKDDPKNYHETDVEVDPNAELSGVYRYIGAGGTVERPTQEGPAMMFNNVKGFPDTRVLTGLMASRRRVGKMFHHDYQTLGQYLNDAVSNPVAPETVAEKDAPAHEVIYKSTDEGFDIRKLVAAPTNTPQDAGPYITVGVVFGSSMDKSKSDVTIHRMVLEDKDKLGIYIMPGGRHIGAFAEEYEKANKPMPITINIGLDPAITIGATFEPPTTPFGYNELGVAGAIRNQAVQLVDGVTVDEKAIARSEYTLEGYIMPNERIQEDINTHTGKAMPEFPGYDGDANPALQVIKVTAVTHRENAIMQSVIGPSEEHVSMAGIPTEASILQLVNRAIPGKVTNVYNPPAGGGKLMTIMQIHKDNEADEGIQRQAALLAFSAFKELKTVILVDEDVDIFDMNDVIWTMNTRFQADQDLMVLSGMRNHPLDPSERPQYDPKSIRFRGMSSKLVIDGTVPYDMKDQFERAQFMKVDDWEKYLK

SEQ ID NO: 13 - DNA, 1473 bp, synthetic construct
  source 1..1473: mol_type = other DNA
  CDS 1..1473: protein_id = 14; translation = MAEQPWDLRRVLDEIKDDPKNYHETDVEVDPNAELSGVYRYIGA GGTVERPTQEGPAMMFNNVKGFPDTRVLTGLMASRRRVGKMFHHDYQTLGQYLNDAVS NPVAPETVAEKDAPAHEVIYKSTDEGFDIRKLVAAPTNTPQDAGPYITVGVVFGSSMD KSKSDVTIHRMVLEDKDKLGIYIMPGGRHIGAFAEEYEKANKPMPITINIGLDPAITI GATFEPPTTPFGYNELGVAGAIRNQAVQLVDGVTVDEKAIARSEYTLEGYIMPNERIQ EDINTHTGKAMPEFPGYDGDANPALQVIKVTAVTHRENAIMQSVIGPSEEHVSMAGIP TEASILQLVNRAIPGKVTNVYNPPAGGGKLMTIMQIHKDNEADEGIQRQAALLAFSAF KELKTVILVDEDVDIFDMNDVIWTMNTRFQADQDLMVLSGMRNHPLDPSERPQYDPKS IRFRGMSSKLVIDGTVPYDMKDQFERAQFMKVDDWEKYLK
atggcagaacagccgtgggatctgcgtcgtgttctggatgaaattaaagatgatccgaagaactatcacgaaaccgatgttgaagttgatccgaatgcagaactgagcggtgtttatcgttatattggtgccggtggcaccgttgaacgtccgacacaagagggtcctgcaatgatgtttaataacgttaaaggttttccggatacacgtgttctgaccggtctgatggcaagccgtcgtcgtgttggtaaaatgtttcatcatgattatcagaccctgggccagtatctgaatgatgcagttagcaatccggttgcaccggaaaccgttgcagaaaaagatgcaccggcacatgaagttatctataaaagcaccgatgaaggcttcgatattcgtaaactggttgcagcaccgaccaatacaccgcaggatgcaggtccgtatattaccgttggtgttgtttttggtagcagcatggataaaagcaaatccgatgttaccattcatcgtatggtgctggaagataaagataaactgggcatctatattatgcctggtggtcgtcatattggcgcatttgccgaagaatatgaaaaagccaataaaccgatgccgatcaccattaacattggtctggatccggcaattaccattggtgcaacctttgaaccgcctaccacaccgtttggttataatgaactgggtgttgcgggtgcaattcgtaatcaggcagttcagctggttgatggtgttaccgttgatgagaaagcaattgcacgtagcgaatataccctggaaggttatatcatgccgaatgaacgtatccaagaagatattaacacccataccggtaaagcaatgccggaatttccgggttatgatggtgatgcaaatccggcactgcaggttattaaagttaccgcagttacccatcgtgaaaatgcaattatgcagagcgttattggtccgagcgaagaacatgttagcatggcaggtattccgaccgaagcaagcattctgcagctggttaatcgtgcaattccgggtaaagtgaccaatgtgtataatccgcctgccggtggtggtaaactgatgaccatcatgcagattcacaaagataatgaagcggatgaaggtattcagcgtcaggcagcactgctggcatttagcgcatttaaagaactgaaaaccgtgatcctggtggatgaagatgtggatattttcgatatgaacgatgtgatctggaccatgaatacccgttttcaggcagatcaggatctgatggttctgagcggtatgcgtaatcatccgctggatccgtcagaacgtccgcagtatgatcctaaaagcattcgttttcgtggtatgagcagcaaactggttattgatggtacagttccgtatgacatgaaagatcagtttgaacgtgcgcagtttatgaaagttgatgactgggagaaatatctgaaataa

SEQ ID NO: 14 - protein, 490 aa, synthetic construct; identical to SEQ ID NO: 12

SEQ ID NO: 15 - DNA, 1476 bp, Companilactobacillus farciminis
  source 1..1476: mol_type = other DNA
  CDS 1..1476: protein_id = 16; translation = MSEQPYDLRRVIDELKQQPGQYHETDVEVDPDAELSGVYRYIGA GGTVKRPTQEGPAMMFNNVKGFPNTRVLIGAMASRKRDGMILHHDYKTLGRLLKDSVE HPVAPEMVDSDKAPVQEVVHKVTDKDFDIRKILPAPTNTEYDAGPYITMGLVLGSDPD KTMTDVTIHRMVLEDKDTIGMYIMPGGRHIGHFQKQFEKLDKPMPITINIGLDPAITI GATFEPPTTPLGYDELNIAGALRNQPVQLVNATSVDEKAIARAEYVVEAEIMPNQTMQ EDINTHTGKAMPEFPGYNGDANPAVNVVKVKAITHRKDNPIMQTTIGPSEEHVSMAGI PTEASILELVDKAIPGKVVNVYNPPAGGGKLMTIMQIHKDNEADEGIQRQAAILALSA FKELKTVFLVDEDVDIFDMNDVVWTMNTRFQGDKDIMVLPGMRNHPLDPSERPEYDPK SIRVRGMSSKTIIDGTVPFDMKDQFIRASFKEVKDWKKYLD
atgtcagagcaaccatatgatttaagaagagttattgatgaactcaaacagcaacctggtcaatatcacgaaactgatgttgaggtcgatcccgatgctgaactatccggagtttaccgttatatcggtgccggtggaacagttaagcgtccaactcaagaaggacctgctatgatgttcaacaatgtgaaaggatttcctaatacacgtgttttgattggtgcaatggctagtcgtaaacgtgacggaatgattttacaccacgattacaagactttaggacgtttgttgaaggattcagtcgaacatcctgtagctccagaaatggtggattctgacaaagctccagttcaagaagtagttcacaaagtaactgataaggatttcgatattagaaagattttgccagctccaactaatactgaatatgatgctggtccttatatcacaatgggtctagtgttaggttcagatccagacaaaaccatgactgatgttacaattcaccgtatggttttggaagataaagatactatcggtatgtacattatgcctggtggtcgtcatattggtcatttccaaaagcaatttgaaaaattagataaaccaatgccaattacaattaatattggtttggatcctgctattactattggtgcaacatttgaaccacctacaacaccacttggctatgatgaattgaatattgccggggctttgcgtaatcaaccagtacaattagttaacgctacatcagttgatgaaaaagcaattgcacgtgctgaatacgtcgttgaagctgaaatcatgcctaatcaaacgatgcaagaagatatcaacactcatactggtaaagccatgccagaatttcctggttacaatggtgatgctaacccagctgttaatgttgttaaggtcaaggccattactcacagaaaagacaatccaattatgcaaacaacaattggacctagtgaagaacacgtttcaatggctggtattcctactgaagcaagtatccttgaattagttgataaggctattcctggaaaagttgttaatgtttacaatcctcctgcaggtggtggtaaattgatgactatcatgcaaatccacaaggataacgaagcagatgaaggtattcaaagacaagctgctatcttagctttatcagcttttaaggaattgaagacggtcttcttagtagatgaagatgttgatatctttgacatgaacgacgttgtttggacgatgaatactcgtttccaaggtgataaagatatcatggttctaccaggaatgcgtaatcatccactcgatccttcagaaagaccagaatatgatcctaagtcaattcgtgttcgtgggatgagttctaagactattatcgatgggacagttccatttgatatgaaagatcaattcatcagagcttcgtttaaagaagtgaaagactggaagaaatatttagattaa

SEQ ID NO: 16 - protein, 491 aa, Companilactobacillus farciminis
MSEQPYDLRRVIDELKQQPGQYHETDVEVDPDAELSGVYRYIGAGGTVKRPTQEGPAMMFNNVKGFPNTRVLIGAMASRKRDGMILHHDYKTLGRLLKDSVEHPVAPEMVDSDKAPVQEVVHKVTDKDFDIRKILPAPTNTEYDAGPYITMGLVLGSDPDKTMTDVTIHRMVLEDKDTIGMYIMPGGRHIGHFQKQFEKLDKPMPITINIGLDPAITIGATFEPPTTPLGYDELNIAGALRNQPVQLVNATSVDEKAIARAEYVVEAEIMPNQTMQEDINTHTGKAMPEFPGYNGDANPAVNVVKVKAITHRKDNPIMQTTIGPSEEHVSMAGIPTEASILELVDKAIPGKVVNVYNPPAGGGKLMTIMQIHKDNEADEGIQRQAAILALSAFKELKTVFLVDEDVDIFDMNDVVWTMNTRFQGDKDIMVLPGMRNHPLDPSERPEYDPKSIRVRGMSSKTIIDGTVPFDMKDQFIRASFKEVKDWKKYLD

SEQ ID NO: 17 - DNA, 1476 bp, synthetic construct
  source 1..1476: mol_type = other DNA
  CDS 1..1476: protein_id = 18; translation = MSEQPYDLRRVIDELKQQPGQYHETDVEVDPDAELSGVYRYIGA GGTVKRPTQEGPAMMFNNVKGFPNTRVLIGAMASRKRDGMILHHDYKTLGRLLKDSVE HPVAPEMVDSDKAPVQEVVHKVTDKDFDIRKILPAPTNTEYDAGPYITMGLVLGSDPD KTMTDVTIHRMVLEDKDTIGMYIMPGGRHIGHFQKQFEKLDKPMPITINIGLDPAITI GATFEPPTTPLGYDELNIAGALRNQPVQLVNATSVDEKAIARAEYVVEAEIMPNQTMQ EDINTHTGKAMPEFPGYNGDANPAVNVVKVKAITHRKDNPIMQTTIGPSEEHVSMAGI PTEASILELVDKAIPGKVVNVYNPPAGGGKLMTIMQIHKDNEADEGIQRQAAILALSA FKELKTVFLVDEDVDIFDMNDVVWTMNTRFQGDKDIMVLPGMRNHPLDPSERPEYDPK SIRVRGMSSKTIIDGTVPFDMKDQFIRASFKEVKDWKKYLD
atgagcgagcagccgtatgatctgcgtcgtgttattgatgaactgaaacagcagcctggtcagtatcatgaaaccgatgttgaagttgatccggatgcagaactgagcggtgtttatcgttatattggtgccggtggcaccgttaaacgtccgacacaagagggtcctgcaatgatgtttaataacgttaaaggctttccgaatacgcgtgttctgattggtgcaatggcaagccgtaaacgtgatggtatgattctgcatcatgattacaaaaccctgggtcgtctgctgaaagatagcgttgaacatccggttgcaccggaaatggttgatagcgataaagcaccggttcaagaagttgttcataaagtcaccgataaggatttcgacatccgtaaaattctgcctgcaccgaccaataccgaatatgatgcaggtccgtatattaccatgggtttagttctgggtagcgatccggataaaaccatgaccgatgttaccattcatcgtatggttctggaagataaagataccatcggcatgtatattatgcctggtggtcgtcatattggccattttcagaaacagtttgagaaactggataaaccgatgccgattaccattaacattggtctggatccggcaattaccattggtgccacctttgaaccgcctaccacaccgctgggttatgatgagctgaatattgccggtgcactgcgtaatcagccggttcagctggttaatgcaaccagcgttgatgaaaaagcaattgcacgtgccgaatatgttgttgaagcagaaattatgccgaaccagaccatgcaagaagatattaacacccataccggtaaagcaatgccggaatttccgggttataatggtgatgcaaatccggcagttaatgtggttaaagttaaagcgattacccaccgcaaagataatccgattatgcagaccaccattggtccgagcgaagaacatgttagcatggcaggtattccgaccgaagcaagcattctggaactggtggataaagccattccgggtaaagttgtgaatgtttataatccgcctgccggtggtggtaaactgatgaccatcatgcagattcacaaagataacgaagccgatgaaggtattcagcgtcaggcagcaattctggcactgagcgcatttaaagaactgaaaaccgtttttctggtggatgaggatgtggatatcttcgatatgaatgatgttgtgtggaccatgaatacccgttttcagggtgataaagacattatggtgctgcctggtatgcgcaatcatccgctggatccgagtgaacgtccggaatatgatccgaaaagcattcgtgttcgtggtatgagcagcaaaaccattattgatggtacagtgccgttcgacatgaaagatcagtttattcgtgccagcttcaaagaggttaaagattggaaaaagtatctggattaa

SEQ ID NO: 18 - protein, 491 aa, synthetic construct; identical to SEQ ID NO: 16

(19) United States
(12) Patent Application Publication
(10) Pub. No.: US 2025/0197895 A1
(43) Pub. Date: Jun. 19, 2025

(54) METHOD FOR PRODUCING METHACRYLIC ACID

(71) Applicant: RIKEN, Wako-shi, Saitama (JP)

(72) Inventors: Tomokazu SHIRAI, Wako-shi, Saitama (JP); Yutaro MORI, Wako-shi, Saitama (JP)

(73) Assignee: RIKEN, Wako-shi, Saitama (JP)

(21) Appl. No.: 18/851,231

(22) PCT Filed: Mar. 28, 2023
(86) PCT No.: PCT/JP2023/012614

§ 371 Date: Sep. 26, 2024

(30) Foreign Application Priority Data
Mar. 29, 2022 (JP) 2022-054328

(51) Int. Cl. C12P 7/40; C12N 9/88; C12N 15/70

(52) U.S. Cl. CPC C12P 7/40; C12N 9/88; C12N 15/70; C12Y 401/01063; C12N 2800/101

(57) ABSTRACT

A method for producing methacrylic acid, comprising: decarboxylating mesaconic acid or an isomer thereof in the presence of protocatechuate decarboxylase.

### TECHNICAL FIELD

The present invention relates to a method for producing methacrylic acid, and more particularly to a method for producing methacrylic acid by decarboxylating mesaconic acid or an isomer thereof using protocatechuate decarboxylase. The present invention also relates to a variant of protocatechuate decarboxylase that can be used in the production, DNA encoding the variant, a vector containing the DNA, and a host cell into which the DNA or the vector is introduced. Furthermore, the present invention relates to a method for producing the variant. Still further, the present invention relates to an agent for promoting the production of methacrylic acid containing the variant and the like.

### BACKGROUND ART

Methacrylic acid is a corrosive liquid with a pungent odor. Industrially, it is widely used in the form of esters, not only as a raw material for synthetic polymers such as acrylic resin, but also for paints, adhesives, paint solvents, and the like. Acrylic resin has a high demand in a wide range of fields such as signboards, lighting equipment, automobile parts, and building materials as a useful material that can replace glass because of its high transparency and excellent weather resistance.

As a method for chemically producing a methacrylic acid derivative, an ACH method using hydrocyanic acid and acetone as raw materials via acetone cyanohydrin (ACH), a C4 oxidation method using isobutylene or tert-butanol as a raw material, and the like are used.

However, these conventional chemical production methods rely on fossil resources. Therefore, in recent years, from the viewpoint of global warming prevention and environmental protection, biosynthetic methods using biological resources as a carbon source to replace conventional fossil resources have attracted attention.

However, many of the currently proposed methods require several steps to obtain methacrylic acid from biological resources, and there are problems that they are complicated and consume a large amount of energy (NPL 1). In this respect, a reaction for producing methacrylic acid from microorganism-derived mesaconic acid in one step has been suggested (PTL 1). However, the enzyme involved in the production has not been clarified, and an efficient method for producing methacrylic acid has not yet been developed.

### CITATION LIST

### Patent Literature

[PTL 1] Japanese Unexamined Patent Application Publication No. 2014-518613

### Non Patent Literature

[NPL 1] Juliana Lebeau, John P Efromson, Michael D Lynch, Front Bioeng Biotechnol. 2020; 8:207.

### SUMMARY OF INVENTION

### Technical Problem

The present invention has been made in view of the above-described problems of the related art, and an object thereof is to provide a method capable of producing methacrylic acid from a carbon source.

### Solution to Problem

As a result of intensive studies conducted to achieve the above object, the present inventors have found that protocatechuate decarboxylase derived from multiple bacterial species has a catalytic activity of decarboxylating mesaconic acid to produce methacrylic acid.

Furthermore, amino acid substitutions were introduced into multiple sites in protocatechuate decarboxylase, and the catalytic activity was evaluated. As a result, it was clarified that the catalytic activity was improved at least 2-fold by substituting histidine at position 327 of the enzyme with a polar neutral amino acid (for example, asparagine, glutamine, serine, threonine, or tyrosine). Furthermore, as a result of substituting the site with a hydrophobic amino acid and evaluating the catalytic activity, it was found that the catalytic activity was also improved at least 2-fold by substituting the site with methionine, phenylalanine, isoleucine, valine, or leucine. In particular, it was clarified that the catalytic activity was improved by 60 times or more by substituting the 327th position of protocatechuate decarboxylase with methionine or phenylalanine.

Furthermore, as a result of introducing further substitutions at various other sites in protocatechuate decarboxylase in addition to the substitution at position 327 and evaluating the catalytic activity, it was found that the catalytic activity was further improved by further amino acid substitution at position 185, position 331, or position 298, and the present invention has thus been completed.

That is, the present invention provides the following aspects.

[1]A method for producing methacrylic acid, comprising: decarboxylating mesaconic acid or an isomer thereof in the presence of protocatechuate decarboxylase.
[2] The production method according to [1], wherein the decarboxylation is performed in a cell expressing protocatechuate decarboxylase, and includes culturing the cell and collecting methacrylic acid produced in the cell and/or a culture thereof.
[3] The production method according to [1] or [2], wherein the protocatechuate decarboxylase is a protein having an amino acid sequence with 90% or more identity to an amino acid sequence represented by SEQ ID NO: 2, 8, 12, or 16.
[4] The production method according to [1] or [2], wherein the protocatechuate decarboxylase is a protocatechuate decarboxylase in which an amino acid at position 327 of an amino acid sequence represented by SEQ ID NO: 2 or an amino acid corresponding to the site is modified to methionine, phenylalanine, asparagine, isoleucine, glutamine, serine, valine, threonine, tyrosine, or leucine.
[5] The production method according to [1] or [2], wherein the protocatechuate decarboxylase is a protocatechuate decarboxylase in which an amino acid at position 327 of an amino acid sequence represented by SEQ ID NO: 2 or an amino acid corresponding to the site is modified to methionine, phenylalanine, asparagine, isoleucine, glutamine, serine, valine, threonine, tyrosine, or leucine, and at least one amino acid modification described in the following (a) to (e) is performed:
(a) modifying an amino acid at position 185 of the amino acid sequence represented by SEQ ID NO: 2 or an amino acid corresponding to the site to valine, isoleucine, methionine, threonine, serine, asparagine, leucine, tyrosine, histidine, or glutamine;
(b) modifying an amino acid at position 331 of the amino acid sequence represented by SEQ ID NO: 2 or an amino acid corresponding to the site to valine or isoleucine;
(c) modifying an amino acid at position 298 of the amino acid sequence represented by SEQ ID NO: 2 or an amino acid corresponding to the site to valine, glutamine, isoleucine, alanine, or leucine;
(d) modifying an amino acid at position 183 of the amino acid sequence represented by SEQ ID NO: 2 or an amino acid corresponding to the site to leucine, tyrosine, glutamic acid, or methionine;
(e) modifying an amino acid at position 438 of the amino acid sequence represented by SEQ ID NO: 2 or an amino acid corresponding to the site to isoleucine, methionine, valine, or threonine.
   [6]A protocatechuate decarboxylase in which an amino acid at position 327 of an amino acid sequence represented by SEQ ID NO: 2 or an amino acid corresponding to the site is modified to methionine, phenylalanine, asparagine, isoleucine, glutamine, serine, valine, threonine, tyrosine, or leucine, and the protocatechuate decarboxylase has a catalytic activity of producing methacrylic acid from mesaconic acid or an isomer thereof.
   [7]A protocatechuate decarboxylase in which an amino acid at position 327 of an amino acid sequence represented by SEQ ID NO: 2 or an amino acid corresponding to the site is modified to methionine, phenylalanine, asparagine, isoleucine, glutamine, serine, valine, threonine, tyrosine, or leucine, and at least one amino acid modification described in the following (a) to (e) is performed:
(a) modifying an amino acid at position 185 of the amino acid sequence represented by SEQ ID NO: 2 or an amino acid corresponding to the site to valine, isoleucine, methionine, threonine, serine, asparagine, leucine, tyrosine, histidine, or glutamine;
(b) modifying an amino acid at position 331 of the amino acid sequence represented by SEQ ID NO: 2 or an amino acid corresponding to the site to valine or isoleucine;
(c) modifying an amino acid at position 298 of the amino acid sequence represented by SEQ ID NO: 2 or an amino acid corresponding to the site to valine, glutamine, isoleucine, alanine, or leucine;
(d) modifying an amino acid at position 183 of the amino acid sequence represented by SEQ ID NO: 2 or an amino acid corresponding to the site to leucine, tyrosine, glutamic acid, or methionine;
(e) modifying an amino acid at position 438 of the amino acid sequence represented by SEQ ID NO: 2 or an amino acid corresponding to the site to isoleucine, methionine, valine, or threonine.
   [8] DNA encoding the protocatechuate decarboxylase according to [6] or [7].
   [9]A vector comprising: the DNA according to [8].
   [10]A host cell into which the DNA according to [8] or a vector including the DNA is introduced.

[11]A method for producing protocatechuate decarboxylase, comprising: culturing the host cell according to [10] and collecting a protein expressed in the host cell.

[12] A method for producing protocatechuate decarboxylase having enhanced catalytic activity of producing methacrylic acid from mesaconic acid or an isomer thereof, the method comprising: modifying an amino acid at position 327 of an amino acid sequence represented by SEQ ID NO: 2 or an amino acid corresponding to the site to methionine, phenylalanine, asparagine, isoleucine, glutamine, serine, valine, threonine, tyrosine, or leucine in protocatechuate decarboxylase.

[13] A method for producing protocatechuate decarboxylase having enhanced catalytic activity of producing methacrylic acid from mesaconic acid or an isomer thereof, the method comprising: modifying an amino acid at position 327 of an amino acid sequence represented by SEQ ID NO: 2 or an amino acid corresponding to the site to methionine, phenylalanine, asparagine, isoleucine, glutamine, serine, valine, threonine, tyrosine, or leucine, and further performing at least one amino acid modification described in the following (a) to (e) in protocatechuate decarboxylase:
(a) modifying an amino acid at position 185 of the amino acid sequence represented by SEQ ID NO: 2 or an amino acid corresponding to the site to valine, isoleucine, methionine, threonine, serine, asparagine, leucine, tyrosine, histidine, or glutamine;
(b) modifying an amino acid at position 331 of the amino acid sequence represented by SEQ ID NO: 2 or an amino acid corresponding to the site to valine or isoleucine;
(c) modifying an amino acid at position 298 of the amino acid sequence represented by SEQ ID NO: 2 or an amino acid corresponding to the site to valine, glutamine, isoleucine, alanine, or leucine;
(d) modifying an amino acid at position 183 of the amino acid sequence represented by SEQ ID NO: 2 or an amino acid corresponding to the site to leucine, tyrosine, glutamic acid, or methionine;
(e) modifying an amino acid at position 438 of the amino acid sequence represented by SEQ ID NO: 2 or an amino acid corresponding to the site to isoleucine, methionine, valine, or threonine.
   [14] An agent for decarboxylating mesaconic acid or an isomer thereof and promoting production of methacrylic acid, comprising: protocatechuate decarboxylase, DNA encoding the protocatechuate decarboxylase, or a vector into which the DNA is inserted.
   [15] The agent according to [14], wherein the protocatechuate decarboxylase is a protein having an amino acid sequence with 90% or more identity to an amino acid sequence represented by SEQ ID NO: 2, 8, 12, or 16.
   [16] The agent according to [14], wherein an amino acid at position 327 of an amino acid sequence represented by SEQ ID NO: 2 or an amino acid corresponding to the site in the protocatechuate decarboxylase is methionine, phenylalanine, asparagine, isoleucine, glutamine, serine, valine, threonine, tyrosine, or leucine.
   [17] The agent according to [14], wherein an amino acid at position 327 of an amino acid sequence represented by SEQ ID NO: 2 or an amino acid corresponding to the site in the protocatechuate decarboxylase is methionine, phenylalanine, asparagine, isoleucine, glutamine, serine, valine, threonine, tyrosine, or leucine, and at least one amino acid described in the following (a) to (e) is included:
(a) an amino acid at position 185 of the amino acid sequence represented by SEQ ID NO: 2 or an amino acid corresponding to the site is valine, isoleucine, methionine, threonine, serine, asparagine, leucine, tyrosine, histidine, or glutamine;
(b) an amino acid at position 331 of the amino acid sequence represented by SEQ ID NO: 2 or an amino acid corresponding to the site is valine or isoleucine;
(c) an amino acid at position 298 of the amino acid sequence represented by SEQ ID NO: 2 or an amino acid corresponding to the site is valine, glutamine, isoleucine, alanine, or leucine;
(d) an amino acid at position 183 of the amino acid sequence represented by SEQ ID NO: 2 or an amino acid corresponding to the site is leucine, tyrosine, glutamic acid, or methionine;
(e) an amino acid at position 438 of the amino acid sequence represented by SEQ ID NO: 2 or an amino acid corresponding to the site is isoleucine, methionine, valine, or threonine.

### Advantageous Effects of Invention

According to the present invention, it becomes possible to produce methacrylic acid from a carbon source. In particular, according to the present invention, it becomes possible to efficiently produce methacrylic acid directly from biological and other resources as a carbon source in one step.

### BRIEF DESCRIPTION OF DRAWINGS

FIG. 1 is a graph showing the results of analyzing the catalytic activity of producing methacrylic acid for protocatechuate decarboxylase derived from Klebsiella pneumoniae (Kp), Bacillus sp. (Bs), Lactiplantibacillus pentosus (Lp), or Companilactobacillus farciminis (Cf).

### DESCRIPTION OF EMBODIMENTS

### <Method 1 for Producing Methacrylic Acid>

As shown in Examples described later, the present inventors have found that protocatechuate decarboxylase (PDC) has a catalytic activity of promoting the reaction of decarboxylating mesaconic acid to produce methacrylic acid (also referred to as “catalytic activity of producing methacrylic acid”). Therefore, the present invention provides a method for producing methacrylic acid, including decarboxylating mesaconic acid or an isomer thereof in the presence of PDC.

“Mesaconic acid,” which is a substrate in the present invention, is a compound also referred to as (2E)-2-methyl-2-butenedioic acid. The “isomer” thereof is not particularly limited, and examples thereof include citraconic acid ((2Z)-2-methyl-2-butenedioic acid, 2-methylmaleic acid) and itaconic acid (2-propene-1,2-dicarboxylic acid, 2-methylidenebutanedioic acid, 2-methylenesuccinic acid). As shown in Examples described later, these compounds can be purchased as, for example, commercially available products. In the present invention, methacrylic acid (2-methylprop-2-enoic acid) is obtained by removing (decarboxylating) one carboxyl group in these dicarboxylic acid compounds.

In the present invention, the conditions for decarboxylating mesaconic acid or an isomer thereof in the presence of PDC are not particularly limited as long as the decarboxylation is promoted and methacrylic acid is produced; those skilled in the art can appropriately adjust and set the composition of the reaction solution, the pH of the reaction solution, the reaction temperature, the reaction time, and the like. In this case, the PDC to be used may be only one type, but may be two or more types.

For example, the reaction solution to which PDC and its substrate, mesaconic acid or an isomer thereof, are added is not particularly limited as long as it does not hinder the reaction, but a buffer solution having a pH of 6 to 10 is preferable, a buffer solution having a pH of 6.5 to 9.5 is more preferable, and a buffer solution having a pH of 6 to 7 (for example, a buffer solution containing potassium chloride and sodium phosphate) is further preferable. Furthermore, from the viewpoint of facilitating the promotion of the reaction, prenylated flavin mononucleotide (prFMN) or an isomer thereof (prFMN^{ketimine}, prFMN^{iminium}, regarding these prFMN and isomers thereof, Karl A. P. Payne et al., Nature, published on Jun. 25, 2015, Vol. 522, No. 7557, pp. 497-501) is preferably included.

The reaction temperature is not particularly limited as long as it does not hinder the reaction, but is usually 10 to 60° C., preferably 10 to 50° C. (for example, 25 to 37° C.). Furthermore, the reaction time is not particularly limited as long as it is a time during which methacrylic acid can be produced, but is usually 30 minutes to 7 days, preferably 12 hours to 2 days.

The methacrylic acid produced under such conditions can be isolated from other components and collected (recovered) by known techniques. Such known techniques are not particularly limited, but examples thereof include a solvent extraction method (for example, continuous liquid-liquid extraction), a pervaporation method, a membrane filtration method, a membrane separation method, a reverse osmosis method, an electrodialysis method, distillation, crystallization, centrifugation, extraction filtration, chromatography (for example, gas chromatography, ion exchange chromatography, size exclusion chromatography, adsorption chromatography), and ultrafiltration. In addition, these methods for collecting methacrylic acid may be performed alone or in appropriate combination in multiple steps.

### <Method 2 for Producing Methacrylic Acid>

As shown in Examples described later, methacrylic acid was successfully produced by culturing a host cell transformed to express PDC.

Therefore, the present invention also provides a method for producing methacrylic acid, including culturing a cell expressing PDC, decarboxylating mesaconic acid or an isomer thereof in the presence of the enzyme in the cell, and collecting methacrylic acid produced in the cell and/or a culture thereof.

The “cell expressing PDC” will be described later; the PDC expressed in such a cell may be only one type, but may be two or more types. The culture conditions for the cells are as described later, but the medium preferably contains mesaconic acid or an isomer thereof, which is a substrate for PDC in the present invention. The culture temperature can be appropriately changed according to the type of host cell to be used, but is usually 20 to 40° C., preferably to 37° C.

In the present invention, the “culture” refers to a medium containing proliferated cells, secretory products of the cells, metabolic products of the cells, and the like, obtained by culturing host cells in a medium, and dilutions and concentrates thereof.

Collection of methacrylic acid from such cells and/or cultures is not particularly limited and can be performed using the above-described known collection methods. The collection timing is appropriately adjusted according to the type of host cell to be used and may be any time during which methacrylic acid can be produced, but is usually 30 minutes to 7 days, preferably 12 hours to 2 days.

### <Protocatechuate Decarboxylase According to the Present Invention>

Next, protocatechuate decarboxylase used in the above-described methods for producing methacrylic acid and the like of the present invention will be described.

“Protocatechuate decarboxylase (PDC)” is registered as EC number: 4.1.1.63 and means an enzyme that catalyzes the reaction of decarboxylating protocatechuic acid (3,4-dihydroxybenzoic acid) to produce catechol, and is also referred to as 3,4-dihydroxybenzoate carboxy-lyase.

In the present invention, PDC is not particularly limited as long as it has an activity of catalyzing the decarboxylation reaction, and for example, as shown in Examples described later, PDC derived from Klebsiella pneumoniae (for example, the protein specified by UniProtKB-B9A9M6, typically the enzyme containing the amino acid sequence represented by SEQ ID NO: 2), PDC derived from Bacillus sp. (for example, the protein specified by UniProtKB-AOA2A8FJC5, typically the enzyme containing the amino acid sequence represented by SEQ ID NO: 8), PDC derived from Lactiplantibacillus pentosus (for example, the protein specified by UniProtKB-AOA2S9VXY3, typically the enzyme containing the amino acid sequence represented by SEQ ID NO: 12), and PDC derived from Companilactobacillus farciminis (for example, the protein specified by UniProtKB-AOAOH4LCR3, typically the enzyme containing the amino acid sequence represented by SEQ ID NO: 16) can be used. Further, the PDC is not limited to those derived from these bacteria, and examples thereof include proteins corresponding to “3,4-dihydroxybenzoate decarboxylase” or “Protocatechuate decarboxylase” on UNIPROT, more specifically, those derived from Clostridium hydroxybenzoicum (for example, the protein specified by UniProtKB-P86833), those derived from Enterobacter cloacae (for example, the protein specified by UniProtKB-AOA7G3F3G5), those derived from Pseudopedobacter saltans (for example, the protein specified by UniProtKB-A0A2W5F854), those derived from Gilliamella apicola (for example, the protein specified by UniProtKB-A0A1B9JW43), or those derived from Lactobacillus pentosus (for example, the protein specified by UniProtKB-A0A241RRA2).

The PDC according to the present invention may be a homolog of the above-mentioned bacterial-derived PDC such as Klebsiella pneumoniae. The homolog of PDC is not particularly limited as long as it has a catalytic activity of producing methacrylic acid, but for example, the homology or identity with the amino acid sequence represented by SEQ ID NO: 2, 8, 12, or 16 is 15% or more (for example, 16% or more, 17% or more, 18% or more, 19% or more), preferably 20% or more (for example, 30% or more, 40% or more), more preferably 50% or more (for example, 60% or more, 70% or more), even more preferably 80% or more (for example, 85% or more, 86% or more, 87% or more, 88% or more, 89% or more), and even more preferably 90% or more (for example, 91% or more, 92% or more, 93% or more, 94% or more, 95% or more, 96% or more, 97% or more, 98% or more, 99% or more). In the present invention, “identity” means the ratio (%) of the number of amino acids that match between the decarboxylase according to the present invention and the amino acid sequence represented by SEQ ID NO: 2, 8, 12, or 16 to the total number of amino acids of the decarboxylase according to the present invention. “Homology” means the ratio (%) of the number of amino acids that are similar between the decarboxylase according to the present invention and the amino acid sequence represented by SEQ ID NO: 2, 8, 12, or 16 to the total number of amino acids of the decarboxylase according to the present invention. Here, “similar amino acid” refers to a combination of amino acids whose BLOSUM62 substitution score is greater than 0.

It should be understood that a change in the amino acid sequence of a protein can occur in nature due to a change in the nucleotide sequence. Therefore, not only the typical amino acid sequences (for example, the amino acid sequences represented by SEQ ID NO: 2, 8, 12, or 16) but also natural PDC variants are included in the PDC according to the present invention as long as they have a catalytic activity of producing methacrylic acid.

Still further, as shown in Examples described later, the catalytic activity of producing methacrylic acid can be improved by substituting an amino acid at a specific site of PDC with another amino acid. Therefore, the PDC according to the present invention includes “a protein (PDC variant)” comprising an amino acid sequence in which one or more amino acids are substituted, deleted, added, and/or inserted in the amino acid sequence of PDC (for example, the amino acid sequence represented by SEQ ID NO: 2, 8, 12, or 16). Here, “plural” is not particularly limited, but is usually 2 to 300, preferably 2 to 250, more preferably 2 to 200, even more preferably 2 to 150, more preferably 2 to 100, even more preferably 2 to 50, more preferably 2 to 40, even more preferably 2 to 30, more preferably 2 to 20 (for example, 2 to 15), even more preferably 2 to 10 (for example, 2 to 8, 2 to 4, 2). Furthermore, the mutation such as amino acid substitution may be naturally occurring as described above, or may be artificially introduced (modified). “Another amino acid” means an amino acid different from the wild-type amino acid at the specific site.

The mutation to be introduced into PDC is not particularly limited as long as the catalytic activity of producing methacrylic acid is improved as compared with that before the introduction, and examples thereof include substitution of the amino acid at position 327 of the amino acid sequence represented by SEQ ID NO: 2 or the amino acid corresponding to the site with another amino acid.

In the present invention, the “corresponding site” refers to a site that is aligned with a specific site in the amino acid sequence represented by SEQ ID NO: 2 when aligned with the amino acid sequence represented by SEQ ID NO: 2 using amino acid sequence analysis software (GENETYX-MAC, Sequencher, etc.) or BLAST (http://blast.ncbi.nlm.nih.gov/Blast.cgi) (for example, “the amino acid at position 327 of the amino acid sequence represented by SEQ ID NO: 2 or the amino acid corresponding to the site” refers to the amino acid at the site aligned with histidine at position 327 in the amino acid sequence represented by SEQ ID NO: 2).

Examples of “substitution with another amino acid” to be introduced at position 327 or the amino acid corresponding to the site include substitution with an amino acid other than histidine, preferably substitution with a polar neutral amino acid or a hydrophobic amino acid.

Examples of the substitution with the “polar neutral amino acid” include substitution with asparagine, glutamine, serine, threonine, tyrosine, or cysteine, and more preferably substitution with asparagine, glutamine, serine, threonine, or tyrosine. More preferably, substitution with asparagine or glutamine is exemplified, and more preferably, substitution with asparagine is exemplified.

Examples of the substitution with the “hydrophobic amino acid” include substitution with methionine, phenylalanine, isoleucine, valine, or leucine, and more preferably substitution with methionine or phenylalanine.

Furthermore, in the PDC according to the present invention, in addition to the amino acid substitution at position 327, a mutation such as an amino acid substitution may be introduced at one or more other sites. The mutation at such other sites is preferably substitution of at least one site selected from the amino acid at position 185 or the amino acid corresponding to the site, the amino acid at position 331 or the amino acid corresponding to the site, the amino acid at position 298 or the amino acid corresponding to the site, the amino acid at position 183 or the amino acid corresponding to the site, and the amino acid at position 438 or the amino acid corresponding to the site in the amino acid sequence represented by SEQ ID NO: 2 with another amino acid. More specifically,
(a) modifying an amino acid at position 185 of the amino acid sequence represented by SEQ ID NO: 2 or an amino acid corresponding to the site to valine, isoleucine, methionine, threonine, serine, asparagine, leucine, tyrosine, histidine, or glutamine (more preferably valine, isoleucine, methionine, threonine, or serine, even more preferably valine, isoleucine, or methionine);
(b) modifying an amino acid at position 331 of the amino acid sequence represented by SEQ ID NO: 2 or an amino acid corresponding to the site to valine or isoleucine (more preferably valine);
(c) modifying an amino acid at position 298 of the amino acid sequence represented by SEQ ID NO: 2 or an amino acid corresponding to the site to valine, glutamine, isoleucine, alanine, or leucine (more preferably valine, glutamine, or isoleucine, even more preferably valine);
(d) modifying an amino acid at position 183 of the amino acid sequence represented by SEQ ID NO: 2 or an amino acid corresponding to the site to leucine, tyrosine, glutamic acid, or methionine; or
(e) modifying an amino acid at position 438 of the amino acid sequence represented by SEQ ID NO: 2 or an amino acid corresponding to the site to isoleucine, methionine, valine, or threonine.

It is desirable that the amino acid substitution at another site is combined with the modification of the amino acid at position 327 of the amino acid sequence represented by SEQ ID NO: 2 or the amino acid corresponding to the site to methionine.

Whether or not PDC has a catalytic activity of producing methacrylic acid can be determined by, for example, directly measuring the amount of methacrylic acid by gas chromatography-mass spectrometry (GC-MS), as shown in Examples described later. Furthermore, it is also possible to determine whether or not the catalytic activity of producing methacrylic acid is higher than that of PDC by comparing with the amount in wild-type PDC (for example, PDC containing the amino acid sequence represented by SEQ ID NO: 2, 8, 12, or 16).

The methacrylic acid according to the present invention preferably has a catalytic activity of producing methacrylic acid of 2 times or more (for example, 3 times or more, 4 times or more, 5 times or more, 6 times or more, 7 times or more, 8 times or more, 9 times or more), more preferably 10 times or more (for example, 20 times or more, 30 times or more, 40 times or more), even more preferably 50 times or more (for example, 60 times or more, 70 times or more, 80 times or more, 90 times or more), more preferably 100 times or more (for example, 110 times or more, 120 times or more, 130 times or more, 140 times or more), even more preferably 150 times or more (for example, 160 times or more, 170 times or more, 180 times or more, 190 times or more), and more preferably 200 times or more (for example, 210 times or more, 220 times or more) that of PDC containing the amino acid sequence represented by SEQ ID NO: 2.

The PDC according to the present invention may be directly or indirectly added with other compounds. The addition is not particularly limited, and may be addition at the gene level or chemical addition. Further, the site to be added is not particularly limited, and may be either the amino terminus (hereinafter also referred to as “N-terminus”) or the carboxyl terminus (hereinafter also referred to as “C-terminus”) of the PDC according to the present invention, or both. Addition at the gene level is achieved by using a DNA encoding the PDC according to the present invention, to which a DNA encoding another protein is added in the same reading frame. “Another protein” added in this way is not particularly limited, and a polyhistidine (His-) tag protein, a FLAG-tag protein (registered trademark, Sigma-Aldrich), glutathione-S-transferase (GST), or other purification tag protein is preferably used for the purpose of facilitating purification of the PDC according to the present invention, and a fluorescent protein such as GFP or a chemiluminescent protein such as luciferase is preferably used for the purpose of facilitating detection of the PDC according to the present invention. Chemical addition may be covalent bonding or non-covalent bonding. The “covalent bond” is not particularly limited, and examples thereof include an amide bond between an amino group and a carboxyl group, an alkylamine bond between an amino group and an alkyl halide group, a disulfide bond between thiols, and a thioether bond between a thiol group and a maleimide group or an alkyl halide group. Examples of the “non-covalent bond” include a biotin-avidin bond. In addition, when the purpose of the “other compounds” chemically added in this way is to facilitate detection of the PDC according to the present invention, a fluorescent dye such as Cy3 or rhodamine is preferably used.

The PDC according to the present invention may be used in combination with other components. The other components are not particularly limited, and examples thereof include sterilized water, physiological saline, vegetable oil, surfactant, fat, solubilizer, buffering agent, protease inhibitor, and preservative.

### <DNA Encoding PDC According to the Present Invention, and Vector Having the DNA>

Next, DNA encoding PDC according to the present invention and the like will be described. By introducing such DNA, it becomes possible to transform a host cell, produce the PDC according to the present invention in the cell, and thus produce methacrylic acid.

The DNA according to the present invention may be natural DNA, DNA in which mutations are artificially introduced into natural DNA, or DNA comprising an artificially designed nucleotide sequence as long as it encodes the above-described PDC according to the present invention. Furthermore, there are no particular limitations on the form, and examples thereof include, in addition to cDNA, genomic DNA, and chemically synthesized DNA. These DNAs can be prepared by those skilled in the art using conventional means. The genomic DNA can be prepared, for example, by extracting genomic DNA from Klebsiella pneumoniae or the like, preparing a genomic library (a plasmid, phage, cosmid, BAC, PAC, etc. can be used as a vector), developing the library, and performing colony hybridization or plaque hybridization using a probe prepared based on the nucleotide sequence of the PDC gene (for example, the nucleotide sequence represented by SEQ ID NO: 1, 7, 11, or 15). It can also be prepared by preparing a primer specific to the PDC gene and performing PCR using the primer. cDNA can be prepared, for example, by synthesizing cDNA based on mRNA extracted from Klebsiella pneumoniae or the like, inserting the cDNA into a vector such as λZAP to prepare a cDNA library, developing the library, and performing colony hybridization or plaque hybridization as described above, or by performing PCR.

A person skilled in the art can introduce a mutation for substituting position 327 or the like with another amino acid into the DNA prepared in this way as necessary by using a known site-directed mutagenesis method. Examples of the site-directed mutagenesis method include the Kunkel method (Kunkel, T. A., Proc Natl Acad Sci USA, 1985, Vol. 82, No. 2, pp. 488-492) and the SOE (splicing-by-overlap-extension)-PCR method (Ho, S. N., Hunt, H. D., Horton, R. M., Pullen, J. K., and Pease, L. R., Gene, 1989, Vol. 77, pp. 51-59).

A person skilled in the art can also artificially design a nucleotide sequence encoding PDC in which position 327 or the like is substituted with another amino acid, and chemically synthesize the DNA according to the present invention based on the sequence information using an automatic nucleic acid synthesizer.

Furthermore, from the viewpoint of further improving the expression efficiency of PDC according to the present invention encoded by the DNA in a host cell, the DNA according to the present invention can also take the form of DNA encoding PDC according to the present invention in which codons are optimized according to the type of the host cell.

The present invention can also take the form of a vector into which the DNA is inserted so that the above-described DNA can be replicated in a host cell.

The “vector” in the present invention can be constructed based on, for example, a plasmid, which is a self-replicating vector, that is, exists as an independent entity outside the chromosome and its replication does not depend on the replication of the chromosome. The vector may also be one that, when introduced into a host cell, is integrated into the genome of the host cell and replicates together with the chromosome into which it is integrated.

Examples of such a vector include a plasmid and phage DNA. Examples of the plasmid include Escherichia coli-derived plasmids (pET22, pBR322, pBR325, pUC118, pUC119, pUC18, pUC19, etc.), yeast-derived plasmids (YEp13, YEp24, YCp50, etc.), and Bacillus subtilis-derived plasmids (pUB110, pTP5, etc.). Examples of the phage DNA include lambda phages (Charon4A, Charon21A, EMBL3, EMBL4, λgt10, λgt11, λZAP, etc.). Furthermore, if the host cell is insect-derived, an insect virus vector such as a baculovirus, if it is plant-derived, T-DNA, etc., and if it is animal-derived, an animal virus vector such as a retrovirus or an adenovirus vector can also be used as the vector according to the present invention. In addition, conventionally used procedures and methods in the field of genetic engineering can be used for the steps and methods of constructing the vector according to the present invention. For example, in order to insert the DNA according to the present invention into a vector, first, a method of cleaving the purified DNA with an appropriate restriction enzyme, inserting the DNA into the restriction enzyme site or multiple cloning site of an appropriate vector, ligating the DNA to the vector, and the like are employed.

Further, the vector according to the present invention may be in the form of an expression vector containing the DNA in a state capable of expressing the PDC according to the present invention encoded by the DNA in a host cell. The “expression vector” according to the present invention preferably contains, in addition to the DNA, a DNA sequence that controls the expression of the DNA and a genetic marker for selecting a transformed host cell in order to introduce the expression vector into the host cell and express the PDC according to the present invention. Examples of the DNA sequence that controls expression include a promoter, an enhancer, a splicing signal, a polyA addition signal, a ribosome binding sequence (SD sequence), and a terminator. The promoter is not particularly limited as long as it exhibits transcriptional activity in a host cell, and can be obtained as a DNA sequence that controls the expression of a gene encoding a protein of the same species or a different species as the host cell. Further, in addition to the DNA sequence that controls the expression, the expression vector may contain a DNA sequence that induces expression. Examples of the DNA sequence that induces expression include a lactose operon capable of inducing the expression of a downstream gene by the addition of isopropyl-R-D-thiogalactopyranoside (IPTG) when the host cell is a bacterium. The genetic marker in the present invention may be appropriately selected depending on the method for selecting a transformed host cell, and examples thereof include a gene encoding drug resistance and a gene that complements auxotrophy.

Further, the DNA or vector according to the present invention may be used in combination with other components. The other components are not particularly limited, and examples thereof include sterilized water, physiological saline, vegetable oil, surfactant, fat, solubilizer, buffering agent, DNase inhibitor, and preservative.

### <Agent for Promoting Production of Methacrylic Acid>

As described above, by using PDC according to the present invention, DNA encoding the PDC, or a vector into which the DNA is inserted, mesaconic acid or an isomer thereof can be decarboxylated to promote the production of methacrylic acid.

Therefore, the present invention provides an agent for decarboxylating mesaconic acid or an isomer thereof and promoting the production of methacrylic acid, including the PDC, the DNA encoding the PDC, or a vector into which the DNA is inserted.

Such an agent may be any agent containing PDC according to the present invention and the like, but may be used in combination with other components. The other components are not particularly limited, and examples thereof include sterilized water, physiological saline, vegetable oil, surfactant, fat, solubilizer, buffering agent, protease inhibitor, DNase inhibitor, and preservative.

The present invention can also provide a kit containing such an agent. In the kit of the present invention, the agent may be contained in the form of a host cell described later, into which the DNA according to the present invention and the like are introduced and transformed. Furthermore, in addition to such an agent, the kit of the present invention may contain mesaconic acid or an isomer thereof, a host cell for introducing the DNA according to the present invention and the like, a medium for culturing the host cell, instructions for their use, and the like. Further, such instructions are instructions for using the agent and the like of the present invention in the above-described method for producing methacrylic acid. The instructions can include, for example, experimental techniques and experimental conditions of the production method of the present invention, and information on the agent and the like of the present invention (for example, information such as a vector map showing the nucleotide sequence of the vector, sequence information of PDC according to the present invention, the origin and properties of the host cell, and information on culture conditions for the host cell).

### <Cell Expressing PDC According to the Present Invention>

Next, a cell expressing PDC according to the present invention will be described. In the present invention, the cell may be a cell that naturally expresses PDC (for example, Klebsiella pneumoniae, Bacillus sp., Lactiplantibacillus pentosus, Companilactobacillus farciminis, Clostridium hydroxybenzoicum, Enterobacter cloacae, Pseudopedobacter saltans, Gilliamella apicola, Lactobacillus pentosus) or a host cell (transformant) into which the DNA or vector according to the present invention is introduced, as shown in Examples described later.

The host cell into which the DNA or vector according to the present invention is introduced is not particularly limited, and examples thereof include microorganisms (Escherichia coli, budding yeast, fission yeast, Bacillus subtilis, actinomycetes, filamentous fungi, etc.), plant cells, insect cells, and animal cells. From the viewpoint that the microorganism exhibits high proliferative ability in a relatively inexpensive medium in a short time, and thus can contribute to the production of methacrylic acid with high productivity, it is preferable to use a microorganism as a host cell, and it is more preferable to use Escherichia coli.

The host cell into which the DNA or vector according to the present invention is introduced is preferably a cell retaining flavin prenyltransferase from the viewpoint of inducing prenylation of flavin mononucleotide (FMN) and producing prFMN or an isomer thereof that contributes to improvement in the productivity of methacrylic acid.

Introduction of the DNA or vector according to the present invention can also be carried out according to methods commonly used in this field. For example, examples of the method for introducing the DNA or vector into microorganisms such as Escherichia coli include the heat shock method, the electroporation method, the spheroplast method, and the lithium acetate method; examples of the method for introducing the DNA or vector into plant cells include a method using Agrobacterium and the particle gun method; examples of the method for introducing the DNA or vector into insect cells include a method using baculovirus and the electroporation method; and examples of the method for introducing the DNA or vector into animal cells include the calcium phosphate method, the lipofection method, and the electroporation method.

The DNA and the like introduced into the host cell in this way may be retained by being randomly inserted into the genomic DNA in the host cell, may be retained by homologous recombination, or if it is a vector, may be replicated and retained as an independent entity outside the genomic DNA.

### <Method for Producing PDC Variant of the Present Invention>

As shown in Examples described later, a PDC variant can be produced in a host cell by culturing the host cell into which DNA encoding the PDC variant of the present invention and the like are introduced.

Therefore, the present invention can also provide a method for producing a PDC variant, including culturing a host cell into which DNA encoding the PDC variant of the present invention or a vector containing the DNA is introduced, and collecting a protein expressed in the host cell.

In the present invention, the conditions for “culturing the host cell” are not particularly limited as long as the host cell can produce the PDC variant of the present invention; those skilled in the art can appropriately adjust and set the temperature, the presence or absence of air addition, the concentration of oxygen, the concentration of carbon dioxide, the pH of the medium, the culture temperature, the culture time, the humidity, and the like according to the type of the host cell, the medium to be used, and the like.

The medium may contain any substance that can be assimilated by the host cell, and examples of the substance include a carbon source, a nitrogen source, a sulfur source, inorganic salts, a metal, peptone, yeast extract, meat extract, casein hydrolyzate, and serum. Further, the medium may be added with, for example, IPTG for inducing the expression of DNA encoding the PDC variant of the present invention, an antibiotic (for example, ampicillin) corresponding to a drug resistance gene that can be encoded by the vector according to the present invention, or a nutrient (for example, arginine, histidine) corresponding to a gene that complements auxotrophy, which can be encoded by the vector according to the present invention.

Examples of the method for “collecting a protein expressed in the host cell” from the host cell cultured in this way include a method of collecting the host cells from the medium by filtration, centrifugation, and the like, treating the collected host cells by cell lysis, grinding treatment, pressure crushing, or the like, and further purifying and concentrating the protein expressed in the host cells by ultrafiltration treatment, solvent precipitation such as salting out and ammonium sulfate precipitation, chromatography (for example, gel chromatography, ion exchange chromatography, affinity chromatography), or the like. Further, when the aforementioned purification tag protein is added to the PDC variant of the present invention, the PDC variant can also be purified and collected using a substrate to which the tag protein adsorbs. Furthermore, these purification and concentration methods may be performed alone or in appropriate combination in multiple steps.

The PDC variant of the present invention can be produced not only by the biological synthesis but also by using the DNA of the present invention and the like and a cell-free protein synthesis system. The cell-free protein synthesis system is not particularly limited, and examples thereof include wheat germ-derived, Escherichia coli-derived, rabbit reticulocyte-derived, and insect cell-derived synthesis systems. Furthermore, those skilled in the art can chemically synthesize the PDC variant of the present invention using a commercially available peptide synthesizer or the like.

The present invention also provides a method for producing PDC having enhanced catalytic activity of producing methacrylic acid, the method including modifying, in PDC, the amino acid at position 327 of the amino acid sequence represented by SEQ ID NO: 2 or the amino acid corresponding to the site to another amino acid (for example, the polar neutral amino acid or the hydrophobic amino acid described above), and optionally further modifying an amino acid at another site.

The “PDC having enhanced catalytic activity of producing methacrylic acid” refers to PDC having higher catalytic activity of producing methacrylic acid as compared with that before the introduction by introducing a mutation into the amino acid at position 327 of the amino acid sequence represented by SEQ ID NO: 2 or the amino acid corresponding to the site, and optionally further introducing a mutation into the amino acid at the other site. The comparison target is usually PDC derived from various organisms such as Klebsiella pneumoniae described above and natural variants thereof.

The preferred embodiments of the mutation to be introduced into the amino acid at position 327 of the amino acid sequence represented by SEQ ID NO: 2 or the amino acid corresponding to the site, or the amino acid at another site are as described in <Protocatechuate Decarboxylase According to the Present Invention> above.

“Modification of an amino acid to another amino acid” in PDC can be carried out by modifying the encoding DNA. The “modification of DNA” can be appropriately carried out by a method known to those skilled in the art, such as site-directed mutagenesis and a method of chemically synthesizing DNA based on modified sequence information. In addition, “modification of an amino acid to another amino acid” can also be performed by using a chemical peptide synthesis method as described above.

Whether or not the catalytic activity of producing methacrylic acid is enhanced by such mutation introduction can be evaluated by GC-MS analysis or the like as described above.

### EXAMPLES

Hereinafter, the present invention will be described more specifically based on Examples and Comparative Examples, but the present invention is not limited to the following Examples.

### Example 1

<Evaluation of Catalytic Activity for Production of Methacrylic Acid Using Protocatechuate Decarboxylase with Mesaconic Acid as Substrate>

Protocatechuate decarboxylase (hereinafter, also referred to as “PDC”) is known to originally have a catalytic activity for the reaction of producing catechol through a decarboxylation reaction using protocatechuic acid (PCA) as a substrate. The present inventors verified the possibility that PDC has a catalytic activity for the reaction of producing methacrylic acid through a decarboxylation reaction using mesaconic acid as a substrate, as follows. The verification was performed for PDCs derived from the following hosts. In the following table, “homology” indicates the homology of those derived from other hosts to the amino acid sequence of PDC derived from Klebsiella pneumoniae.

TABLE 1

| Host | Abbreviation | Homology |
|---|---|---|
| Klebsiella pneumoniae | Kp | — |
| Bacillus sp. | Bs | 46.8% |
| Companilactobacillus | Cf | 50.9% |
| farciminis | | |
| Lactiplantibacillus | Lp | 46.3% |
| pentosus | | |

### <Preparation of Plasmid Vector Capable of Expressing Protocatechuate Decarboxylase>

First, in order to efficiently express PDC in Escherichia coli, the nucleotide sequence derived from the wild type (Klebsiella pneumoniae, Bacillus sp., Lactiplantibacillus pentosus, or Companilactobacillus farciminis) (the nucleotide sequence represented by SEQ ID NO: 1, 7, 11, or 15) was modified in a form in which a polyhistidine tag was fused to the C-terminus in consideration of the codon usage frequency in Escherichia coli (the nucleotide sequence represented by SEQ ID NO: 3, 9, 13, or 17). Next, DNA comprising the modified nucleotide sequence was chemically synthesized by a conventional method. Then, the DNA thus prepared and a pET22b(+) vector (manufactured by Novagen) were ligated by the Gibson Assembly method (using a kit NEBuilder HiFi DNA Assembly Master Mix (registered trademark) manufactured by New England Biolabs) to prepare a plasmid vector (PDC vector) capable of expressing the wild-type PDC in Escherichia coli.

### <Preparation of Plasmid Vector Capable of Expressing Flavin Prenyltransferase>

Similarly, a plasmid vector (UbiX vector) capable of expressing the wild-type UbiX in Escherichia coli was prepared by ligating DNA obtained by amplifying a gene (the nucleotide sequence represented by SEQ ID NO: 5) encoding flavin prenyltransferase (hereinafter, also referred to as “UbiX”) from an Escherichia coli (K-12) strain by the polymerase chain reaction method and a pCOLADuet vector (manufactured by Novagen) by the Gibson Assembly method.

### <Preparation of Enzyme Reaction Solution and Measurement of Enzyme Activity>

The vectors prepared as described above (5 μg of PDC vector and 5 μg of UbiX vector) were introduced into an Escherichia coli C41(DE3) strain (manufactured by Lucigen Corporation, 100 μL) by the heat shock method to prepare a transformant co-expressing wild-type PDC and UbiX.

The transformant was cultured in an LB medium supplemented with ampicillin and kanamycin for 6 hours. The growth of the transformant reached a plateau by the 6-hour culture (preculture).

An enzyme reaction medium was prepared by adding mesaconic acid (manufactured by Sigma-Aldrich), which is a substrate, to 12 g/L tryptone, 24 g/L yeast extract, 10 g/L glycerol, 9.4 g/L dipotassium hydrogen phosphate, 2.2 g/L potassium dihydrogen phosphate, 20 g/L lactose, 100 mg/L ampicillin, and 50 mg/L kanamycin to a concentration of 5 g/L.

Then, 20 μL of the Escherichia coli culture solution cultured for 6 hours and 1 mL of the enzyme reaction medium were added to a 10 mL vial for a headspace gas chromatography mass spectrometer (HS/GSMS), immediately after which the vial was capped, and further cultured at 37° C. and a shaking speed of 180 rpm. The culture was terminated 72 hours after the start of the culture.

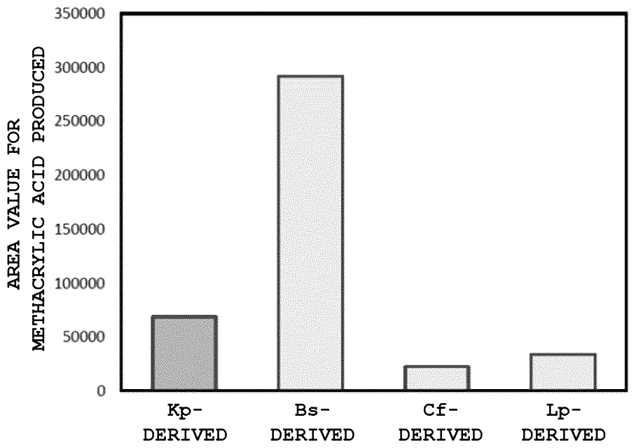

The recovered Escherichia coli culture solution was centrifuged at 150,000 rpm for 15 minutes, and 200 μL of the supernatant was collected in a 1.5 mL tube. Methacrylic acid was extracted from the Escherichia coli culture solution into ethyl acetate by adding 20 μL of 1 M hydrochloric acid and 200 μL of ethyl acetate and shaking at 2,000 rpm for 1 hour. The extraction sample was centrifuged at 150,000 rpm for 15 minutes, after which the upper layer of ethyl acetate was recovered, and methacrylic acid was analyzed by gas chromatography mass spectrometry. The obtained results are shown in FIG. 1. In FIG. 1, the area value of the peak derived from methacrylic acid in the chromatogram obtained by the gas chromatography mass spectrometry is shown.

### Results

As a result of the above analysis, as shown in FIG. 1, methacrylic acid was detected from ethyl acetate. This revealed for the first time that PDC, which has not been known so far, has a catalytic activity of producing methacrylic acid using mesaconic acid as a substrate. In addition, since the production of methacrylic acid was detected in all of the four bacterial species, it is highly probable that the enzyme called protocatechuate decarboxylase can be used as a methacrylic acid-producing enzyme. In these enzymes, the amino acids present in the substrate binding site are almost the same, but in different parts, the amino acid corresponding to alanine at position 185 of the Kp-derived amino acid sequence (amino acid sequence represented by SEQ ID NO: 2) was valine in Bs-derived, which has high catalytic activity among the four (position 187 in the amino acid sequence represented by SEQ ID NO: 8). Therefore, it is suggested that this difference may affect the activity.

### Example 2

### <Preparation and Evaluation of Protocatechuate Decarboxylase Variant>

The present inventors prepared a large number of PDC variants by introducing mutations with amino acid substitutions at various positions of PDC by the methods described below and the like in order to be able to produce methacrylic acid with even higher productivity. The variants were evaluated for catalytic activity for the production of methacrylic acid using mesaconic acid as a substrate.

### <Preparation of Plasmid Vector Capable of Expressing Protocatechuate Decarboxylase Variant>

First, as shown in Table 2 below, in order to introduce mutations with amino acid substitutions into PDC at each of the five sites of PDC, primers encoding amino acid sequences into which each mutation was introduced were designed and synthesized.

TABLE 2

| Amino | Amino Acid | Amino Acid |
|---|---|---|
| Acid | Before | After |
| Position | Substitution | Substitution |
| 188 | R | K, H, S, T, N, Q, A, V, I, L, M, F, Y, W |
| 327 | H | R, K, S, T, N, Q, A, V, I, L, M, F, Y, W |
| 363 | K | R, H, S, T, N, Q, A, V, I, L, M, F, Y, W |
| 394 | E | R, K, H, S, T, N, Q, A, V, I, L, M, F, Y, W |
| 436 | H | R, K, S, T, N, Q, A, V, I, L, M, F, Y, W |

Then, using the PDC vector as a template alongside the primers, a plasmid vector (PDC variant vector) capable of expressing PDC—into which each mutation was introduced in Escherichia coli with a polyhistidine tag fused to the C-terminus—was prepared following the protocol of the Gibson Assembly method.

### <Preparation of Enzyme Reaction Solution and Measurement of Enzyme Activity>

Hereinafter, a method for preparing an enzyme reaction solution, a method for measuring enzyme activity, and the like will be described in detail, but the basic method is the same as the method performed in the verification of the catalytic activity of the above-described wild-type PDC.

The vectors prepared as described above (5 μg of PDC variant vector and 5 μg of UbiX vector) were introduced into an Escherichia coli C41(DE3) strain (manufactured by Lucigen Corporation, 100 μL) by the heat shock method to prepare a transformant co-expressing each PDC variant and UbiX.

Then, these transformants were each cultured in an LB medium supplemented with ampicillin and kanamycin for 6 hours. The growth of these transformants reached a plateau by the 6-hour culture (preculture). Therefore, since the amount of bacterial cells at the start of the enzyme reaction described later becomes uniform between the transformant used in the verification of the catalytic activity of the wild-type PDC and the transformants expressing these PDC variants, it becomes easy to compare and examine the catalytic activities.

An enzyme reaction medium was prepared by adding mesaconic acid (manufactured by Sigma-Aldrich), which is a substrate, to 12 g/L tryptone, 24 g/L yeast extract, 10 g/L glycerol, 9.4 g/L dipotassium hydrogen phosphate, 2.2 g/L potassium dihydrogen phosphate, 20 g/L lactose, 100 mg/L ampicillin, and 50 mg/L kanamycin to a concentration of 5 g/L.

Then, 20 μL of the Escherichia coli culture solution cultured for 6 hours and 1 mL of the enzyme reaction medium were added to a 10 mL vial for a headspace gas chromatography mass spectrometer (HS/GSMS), immediately after which the vial was capped, and further cultured at 37° C. and a shaking speed of 180 rpm. The culture was terminated 72 hours after the start of the culture.

The recovered Escherichia coli culture solution was centrifuged at 150,000 rpm for 15 minutes, and 200 μL of the supernatant was collected in a 1.5 mL tube. Methacrylic acid was extracted from the Escherichia coli culture solution into ethyl acetate by adding 20 μL of 1 M hydrochloric acid and 200 μL of ethyl acetate and shaking at 2,000 rpm for 1 hour. The extraction sample was centrifuged at 150,000 rpm for 15 minutes, after which the upper layer of ethyl acetate was recovered, and methacrylic acid was analyzed by gas chromatography mass spectrometry.

### Results

Table 3 below shows the relative values of the amount of methacrylic acid produced in each PDC variant to that in wild-type PDC, which were calculated based on the obtained peak areas.

TABLE 3

| | |
|---|---|
| WT activity | Mutation Introduction Site |

| | | | | | |
|---|---|---|---|---|---|
| is set to 1.0 | R188 | H327 | K363 | E394 | H436 |

| | | | | | | |
|---|---|---|---|---|---|---|
| Amino Acid | R | WT | 0.1 | 0.7 | 0.7 | 1.1 |
| Residue | K | 0.0 | 0.4 | WT | 0.6 | 1.0 |
| After | H | 0.1 | WT | 0.5 | 1.0 | WT |
| Substitution | S | 0.1 | 2.9 | 0.6 | 1.0 | 0.9 |
| | T | 0.2 | 2.6 | 1.0 | 0.4 | 1.5 |
| | N | 0.1 | 10.7 | 0.8 | 0.6 | 1.2 |
| | Q | 0.3 | 4.1 | 1.2 | 1.1 | 1.5 |
| | A | 0.0 | 1.7 | 0.0 | 0.0 | 0.0 |
| | V | 0.0 | 2.7 | 0.0 | 0.0 | 0.0 |
| | I | 0.0 | 7.7 | 0.0 | 0.0 | 0.0 |
| | L | 0.0 | 2.1 | 0.0 | 0.0 | 0.0 |
| | M | 0.0 | 75.3 | 0.0 | 0.0 | 0.0 |
| | F | 0.0 | 68.1 | 0.0 | 0.0 | 0.0 |
| | Y | 0.4 | 2.2 | 0.4 | 0.8 | 1.1 |
| | W | 0.0 | 0.2 | 0.0 | 0.0 | 0.0 |

As shown in the table, of the five sites into which mutations were introduced, it was clarified that the catalytic activity for the production of methacrylic acid was generally improved at position 327 by substituting histidine at the site with another amino acid (serine, threonine, asparagine, glutamine, tyrosine) (the catalytic activity was improved by at least about 2.2 times compared to wild-type EDO). In particular, when the substitution was asparagine, the catalytic activity for the production of methacrylic acid was improved by 10 times or more (the production amount of methacrylic acid with the substrate mesaconic acid charged at 5 g/L was 2.1 mg/L for the wild type, whereas the production amount of methacrylic acid was 22.5 mg/L for H327N). It was also found that the substitution of position 327 with methionine, phenylalanine, isoleucine, valine, or leucine also improved the activity by at least 2-fold. In particular, it was clarified that the substitution with methionine or phenylalanine improved the catalytic activity by 60 times or more (the production amount of methacrylic acid with the substrate mesaconic acid charged at 5 g/L was 2.1 mg/L for the wild type, whereas the production amount of methacrylic acid was 202 mg/L for H327M).

### Example 3

Further amino acid substitutions were introduced into the PDC single variant (H327M) that showed the highest catalytic activity in Example 2, and methacrylic acid was analyzed for these PDC double variants by the same method as described above. The obtained results are shown in Table 4.

TABLE 4

| WT activity | | Mutation Introduction Site | | | | | | |
|---|---|---|---|---|---|---|---|---|
| is set to 1.0 | | F183 | A185 | N298 | A331 | Y392 | H436 | L438 |
| Amino Acid | A | 0.0 | | 61.5 | | 0.0 | 0.0 | 0.0 |
| Residue | V | 0.0 | 227.7 | 96.7 | 131.9 | 0.0 | 0.0 | 17.6 |
| After | I | 0.0 | 175.8 | 86.1 | 70.3 | 0.0 | 0.0 | 52.7 |
| Substitution | L | 61.5 | 70.3 | 8.8 | 0.0 | 0.0 | 0.0 | |
| | M | 37.1 | 161.7 | 0.0 | 0.0 | 0.0 | 0.0 | 26.4 |
| | F | | 0.0 | 0.0 | 0.0 | 0.0 | 0.0 | 0.0 |
| | Y | 52.7 | 52.7 | 0.0 | 0.0 | | 0.0 | 0.0 |
| | W | 0.0 | 0.0 | 0.0 | 0.0 | 0.0 | 0.0 | 0.0 |
| | R | 0.0 | 0.0 | 0.0 | 0.0 | 0.0 | 0.0 | 0.0 |
| | K | 0.0 | 0.0 | 0.0 | 0.0 | 0.0 | 0.0 | 0.0 |
| | H | 0.0 | 50.1 | 0.0 | 0.0 | 0.0 | | 0.0 |
| | S | 0.0 | 91.4 | 0.0 | 0.0 | 0.0 | 0.0 | 0.0 |
| | T | 0.0 | 114.3 | 0.0 | 0.0 | 0.0 | 0.0 | 8.8 |
| | N | 0.0 | 79.1 | | 0.0 | 0.0 | 0.0 | 0.0 |
| | Q | 0.0 | 44.0 | 87.9 | 0.0 | 0.0 | 0.0 | 0.0 |
| | D | 0.0 | 0.0 | 0.0 | 0.0 | 0.0 | 0.0 | 0.0 |
| | E | 39.3 | 0.0 | 0.0 | 0.0 | 0.0 | 0.0 | 0.0 |

As shown in the table, it was clarified that by further adding the following modifications in addition to the modification of position 327 to methionine, the catalytic activity was improved by at least about 2 times compared to wild-type PDC:
(a) modifying position 185 to valine, isoleucine, methionine, threonine, serine, asparagine, leucine, tyrosine, histidine, or glutamine;
(b) modifying position 331 to valine or isoleucine;
(c) modifying position 298 to valine, glutamine, isoleucine, alanine, or leucine;
(d) modifying position 183 to leucine, tyrosine, glutamic acid, or methionine; or
(e) modifying position 438 to isoleucine, methionine, valine, or threonine.

In particular, it was clarified that by further modifying position 298 to valine, position 331 to valine, or position 185 to valine, isoleucine, or methionine, the catalytic activity was improved by 100 times or more compared to wild-type PDC (the production amount of methacrylic acid with the substrate mesaconic acid charged at 5 g/L was 2.1 mg/L for the wild type, whereas the production amount of methacrylic acid was 202 mg/L for H327M and 525 mg/L for H327M/A185V).

### INDUSTRIAL APPLICABILITY

As described above, according to the present invention, methacrylic acid can be produced by using protocatechuate decarboxylase. Further, according to the present invention, methacrylic acid can be produced by biosynthesis without relying on chemical synthesis, so that the burden on the environment is small. Therefore, the present invention is extremely useful in the production of raw materials for various synthetic polymers such as paints, adhesives, and acrylic resins.

1. A method for producing methacrylic acid, comprising: decarboxylating mesaconic acid or an isomer thereof in the presence of protocatechuate decarboxylase.

2. The production method according to claim 1, wherein the decarboxylation is performed in a cell expressing protocatechuate decarboxylase, and includes culturing the cell and collecting methacrylic acid produced in the cell and/or a culture thereof.

3. The production method according to claim 1, wherein the protocatechuate decarboxylase is a protein having an amino acid sequence with 90% or more identity to an amino acid sequence represented by SEQ ID NO: 2, 8, 12, or 16.

4. The production method according to claim 1, wherein the protocatechuate decarboxylase is a protocatechuate decarboxylase in which an amino acid at position 327 of an amino acid sequence represented by SEQ ID NO: 2 or an amino acid corresponding to the site is modified to methionine, phenylalanine, asparagine, isoleucine, glutamine, serine, valine, threonine, tyrosine, or leucine.

5. The production method according to claim 1, wherein the protocatechuate decarboxylase is a protocatechuate decarboxylase in which an amino acid at position 327 of an amino acid sequence represented by SEQ ID NO: 2 or an amino acid corresponding to the site is modified to methionine, phenylalanine, asparagine, isoleucine, glutamine, serine, valine, threonine, tyrosine, or leucine, and at least one amino acid modification described in the following (a) to (e) is performed:
(a) modifying an amino acid at position 185 of the amino acid sequence represented by SEQ ID NO: 2 or an amino acid corresponding to the site to valine, isoleucine, methionine, threonine, serine, asparagine, leucine, tyrosine, histidine, or glutamine;
(b) modifying an amino acid at position 331 of the amino acid sequence represented by SEQ ID NO: 2 or an amino acid corresponding to the site to valine or isoleucine;
(c) modifying an amino acid at position 298 of the amino acid sequence represented by SEQ ID NO: 2 or an amino acid corresponding to the site to valine, glutamine, isoleucine, alanine, or leucine;
(d) modifying an amino acid at position 183 of the amino acid sequence represented by SEQ ID NO: 2 or an amino acid corresponding to the site to leucine, tyrosine, glutamic acid, or methionine;
(e) modifying an amino acid at position 438 of the amino acid sequence represented by SEQ ID NO: 2 or an amino acid corresponding to the site to isoleucine, methionine, valine, or threonine.

6. A protocatechuate decarboxylase in which an amino acid at position 327 of an amino acid sequence represented by SEQ ID NO: 2 or an amino acid corresponding to the site is modified to methionine, phenylalanine, asparagine, isoleucine, glutamine, serine, valine, threonine, tyrosine, or leucine, and the protocatechuate decarboxylase has a catalytic activity of producing methacrylic acid from mesaconic acid or an isomer thereof.

7. A protocatechuate decarboxylase in which an amino acid at position 327 of an amino acid sequence represented by SEQ ID NO: 2 or an amino acid corresponding to the site is modified to methionine, phenylalanine, asparagine, isoleucine, glutamine, serine, valine, threonine, tyrosine, or leucine, and at least one amino acid modification described in the following (a) to (e) is performed:
(a) modifying an amino acid at position 185 of the amino acid sequence represented by SEQ ID NO: 2 or an amino acid corresponding to the site to valine, isoleucine, methionine, threonine, serine, asparagine, leucine, tyrosine, histidine, or glutamine;
(b) modifying an amino acid at position 331 of the amino acid sequence represented by SEQ ID NO: 2 or an amino acid corresponding to the site to valine or isoleucine;
(c) modifying an amino acid at position 298 of the amino acid sequence represented by SEQ ID NO: 2 or an amino acid corresponding to the site to valine, glutamine, isoleucine, alanine, or leucine;
(d) modifying an amino acid at position 183 of the amino acid sequence represented by SEQ ID NO: 2 or an amino acid corresponding to the site to leucine, tyrosine, glutamic acid, or methionine;
(e) modifying an amino acid at position 438 of the amino acid sequence represented by SEQ ID NO: 2 or an amino acid corresponding to the site to isoleucine, methionine, valine, or threonine.

8. DNA encoding the protocatechuate decarboxylase according to claim 6.

9. A vector comprising: the DNA according to claim 8.

10. A host cell into which the DNA according to claim 8 or a vector including the DNA is introduced.

11. A method for producing protocatechuate decarboxylase, comprising: culturing the host cell according to claim 10 and collecting a protein expressed in the host cell.

12. A method for producing protocatechuate decarboxylase having enhanced catalytic activity of producing methacrylic acid from mesaconic acid or an isomer thereof, the method comprising: modifying an amino acid at position 327 of an amino acid sequence represented by SEQ ID NO: 2 or an amino acid corresponding to the site to methionine, phenylalanine, asparagine, isoleucine, glutamine, serine, valine, threonine, tyrosine, or leucine in protocatechuate decarboxylase.

13. The production method according to claim 12 the method further performing at least one amino acid modification described in the following (a) to (e) in protocatechuate decarboxylase:
(a) modifying an amino acid at position 185 of the amino acid sequence represented by SEQ ID NO: 2 or an amino acid corresponding to the site to valine, isoleucine, methionine, threonine, serine, asparagine, leucine, tyrosine, histidine, or glutamine;
(b) modifying an amino acid at position 331 of the amino acid sequence represented by SEQ ID NO: 2 or an amino acid corresponding to the site to valine or isoleucine;
(c) modifying an amino acid at position 298 of the amino acid sequence represented by SEQ ID NO: 2 or an amino acid corresponding to the site to valine, glutamine, isoleucine, alanine, or leucine;
(d) modifying an amino acid at position 183 of the amino acid sequence represented by SEQ ID NO: 2 or an amino acid corresponding to the site to leucine, tyrosine, glutamic acid, or methionine;
(e) modifying an amino acid at position 438 of the amino acid sequence represented by SEQ ID NO: 2 or an amino acid corresponding to the site to isoleucine, methionine, valine, or threonine.

14. An agent for decarboxylating mesaconic acid or an isomer thereof and promoting production of methacrylic acid, comprising: protocatechuate decarboxylase, DNA encoding the protocatechuate decarboxylase, or a vector into which the DNA is inserted.

15. The agent according to claim 14, wherein the protocatechuate decarboxylase is a protein having an amino acid sequence with 90% or more identity to an amino acid sequence represented by SEQ ID NO: 2, 8, 12, or 16.

16. The agent according to claim 14, wherein an amino acid at position 327 of an amino acid sequence represented by SEQ ID NO: 2 or an amino acid corresponding to the site in the protocatechuate decarboxylase is methionine, phenylalanine, asparagine, isoleucine, glutamine, serine, valine, threonine, tyrosine, or leucine.

17. The agent according to claim 14, wherein an amino acid at position 327 of an amino acid sequence represented by SEQ ID NO: 2 or an amino acid corresponding to the site in the protocatechuate decarboxylase is methionine, phenylalanine, asparagine, isoleucine, glutamine, serine, valine, threonine, tyrosine, or leucine, and at least one amino acid described in the following (a) to (e) is included:
(a) an amino acid at position 185 of the amino acid sequence represented by SEQ ID NO: 2 or an amino acid corresponding to the site is valine, isoleucine, methionine, threonine, serine, asparagine, leucine, tyrosine, histidine, or glutamine;
(b) an amino acid at position 331 of the amino acid sequence represented by SEQ ID NO: 2 or an amino acid corresponding to the site is valine or isoleucine;
(c) an amino acid at position 298 of the amino acid sequence represented by SEQ ID NO: 2 or an amino acid corresponding to the site is valine, glutamine, isoleucine, alanine, or leucine;
(d) an amino acid at position 183 of the amino acid sequence represented by SEQ ID NO: 2 or an amino acid corresponding to the site is leucine, tyrosine, glutamic acid, or methionine;
(e) an amino acid at position 438 of the amino acid sequence represented by SEQ ID NO: 2 or an amino acid corresponding to the site is isoleucine, methionine, valine, or threonine.
